(19) United States
(12) Patent Application Publication
(10) Pub. No.: US 2002/0182232 A9
(43) Pub. Date: Dec. 5, 2002

(54) Cosmetic composition comprising at least one ingredient chosen from compounds of formula (I) and salts thereof, use thereof as colouring agent, and novel compounds of formulae (IIa), (IIIa), (IVa), (Va), and (VIa), and salts thereof

(76) Inventors: Jean-Christophe Henrion, Pantin (FR); Michel Philippe, Wissous (FR)

Correspondence Address: FINNEGAN, HENDERSON, FARABOW, GARRETT &, DUNNER LLP, 1300 I STREET, NW, WASHINGTON, DC, 20005, US

(21) Appl. No.: 09/810,628

(22) Filed: Mar. 19, 2001

(30) Foreign Application Priority Data
Mar. 17, 2000 (FR) 00 03471

(51) Int. Cl. A61K 7/021

(52) U.S. Cl. 424/401; 424/63

(57) ABSTRACT

A cosmetic composition containing at least one ingredient chosen from compounds of formula (I) and salts thereof:

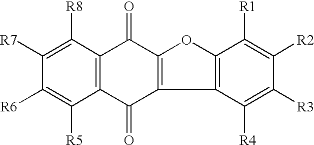

The invention also relates to the use of at least one ingredient chosen from compounds of formula (I) and salts thereof as defined above as a coloring agent, such as in a cosmetic composition, as well as to certain novel furan-naphthoquinone compounds and salts thereof.

The present invention relates to the cosmetic use of compounds derived from furan-naphthoquinones, such as in cosmetic compositions and also to novel compounds.

Cosmetic compositions, such as make-up compositions, such as free powders, compact powders, foundations, face powders, eyeshadows, lipsticks and nail varnishes, contain a suitable vehicle and various colouring agents intended to give the compositions a certain colour before and/or after they are applied to the skin, mucous membranes and/or superficial body growths, such as the nails, the eyelashes and the hair.

To create colours, a relatively limited range of colouring agents is used nowadays, among which mention may be made of compounds that are generally insoluble in aqueous and organic media, such as organic lacquers, mineral pigments and nacreous pigments.

The pigments and lacquers used in the field of make-up are very diverse in origin and chemical nature. Their physicochemical properties, in particular particle size, specific surface area, density, etc., are thus very different. These differences are reflected by variations in behavior: their ease of use; of dispersion in the medium; their light-fastness and heat stability; and their mechanical properties.

Thus, mineral pigments, in particular mineral oxides, such as iron oxides, are very light-fast and pH-stable, but can give rather dull, lifeless and pale colours. Thus, it is necessary to introduce a large amount of them into cosmetic formulations in order to obtain a sufficiently saturated mark. However, the high percentage of mineral particles may affect the sheen of the composition.

In order to obtain coloured effects, nacreous pigments of varied, but never very intense, colours can also be used, thereby giving iridescent but usually rather weak effects.

In the field of temporary or short-term dyeing of the hair, which gives rise to a slight change in the natural colour of the hair, which holds from one shampoo-wash to another and which serves to enhance or correct a shade already obtained, colouring with common mineral pigments in order to give the hair a temporary glint has already been proposed, but the shades obtained by this colouring can remain rather dull, too uniform and lifeless.

In the field of make-up, only organic lakes have hitherto made it possible to obtain bright and intense colours. However, most organic lakes have very poor light-fastness, which is reflected by a very marked attenuation of their colour over time. They may also be unstable with respect to heat and pH. Furthermore, certain lakes can give rise to excessive bleeding, i.e., they have the drawback of staining the support onto which they are applied. Consequently, this may stain contact lenses in the case of eyeliners and mascaras, and leave a coloration on the skin and the nails after removal of make-up in the case of lipsticks and nail varnishes. Finally, the instability of lakes is further exacerbated when they are combined with photo-reactive pigments, such as titanium dioxide. Now, these pigments are very widely used in make-up, especially for protection against UV radiation. Consequently, the use of organic lakes in cosmetics is quite limited, thereby limiting the tints which may be produced.

Thus, the need remains to have available colouring agents which may be used in cosmetics, to obtain a suitable coloration of the compositions and of the make-up obtained, the colouring agents moreover having at least one of the following properties: good covering power, good heat stability, and photochemical stability, while at the same time producing little bleeding.

The inventors have discovered that the use of a specific family of compounds, derived from furan-naphthoquinones, makes it possible, unexpectedly, to obtain at least one of these desirable results.

Thus, a subject of the invention is a cosmetic composition comprising, in a cosmetically acceptable medium, at least one ingredient chosen from compounds of formula (I) and salts thereof:

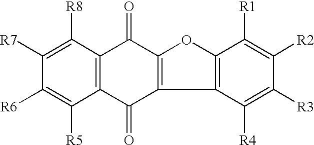

in which the radicals R1 to R8 are as defined below.

A subject of the invention is also the use of at least one colouring agent chosen from compounds of formula (I) and salts thereof as defined below, such as in a cosmetic composition.

Some of the compounds derived from furan-naphthoquinones that are used in the present invention are known in the literature.

Thus, some have been disclosed in particular in the publication by Shand et al., Tetrahedron, 1963, vol. 19, pp. 1919-1937, the disclosure of which is incorporated by reference, which concerns the formation of dibenzofuran derivatives by rearrangement of diquinones.

Others are described in the publication by Sankaram et al., Phytochemistry, 1981, vol. 20, No. 5, pp. 1093-1096, the disclosure of which is incorporated by reference, which concerns pentacyclic quinones extracted from the wood of Diospyros melanoxylon.

Others are also described in the Japanese publication by Ishikawa et al., Nihon Kagakkai-shi, 1988, No. 5, pp. 743-751, the disclosure of which is incorporated by reference, which concerns the synthesis of compounds of the dibenzofuran and dinaphthofuran-quinone type.

However, none of these publications teaches or suggests that these compounds may be used successfully as colouring agents, such as pigments, in cosmetic compositions. The inventors discovered that such a use was possible.

These compounds may provide at least one of the following advantages: good heat stability, pH-stability, and light-fastness.

They can be in solid form and can produce bright and varied colours, depending on the nature of the substituents.

Their colour can be pure and highly saturated, and can cover a very broad range, from yellow to dark red.

Their colouring strength (or saturation loss) and their covering power can also be very good and comparable with those of the pigments of the prior art.

Furthermore, in general, these compounds are insoluble in water and very sparingly soluble in oils of varied nature and/or polarity. Consequently, these compounds can have the advantage of bleeding very little when they are used in compositions comprising fatty substances.

It has also been found that it is moreover possible to modify the colour of the at least one ingredient chosen from compounds of formula (I) and salts thereof by varying the nature and/or position of the various substituents R present on the molecule.

The at least one ingredient according to the invention thus is chosen from compounds of formula (I), and salts thereof,:

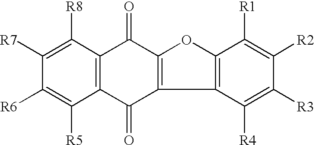

in which the radicals R1 to R8, which may be identical or different, are chosen from:

a hydrogen atom;

a halogen atom, such as chlorine, bromine, iodine and fluorine;

a hydroxyl radical —OH;

an amino radical —NRR′, wherein R and R′, which may be identical or different, are each a unit chosen from a hydrogen atom, and linear, branched and cyclic, saturated and unsaturated hydrocarbon-based radicals comprising from 1 to 36 carbon atoms;

a nitro radical —NO₂;

an alkylamido radical —NH—CO—R″, wherein R″ is a unit chosen from linear, branched and cyclic, saturated and unsaturated hydrocarbon-based radicals comprising from 1 to 36 carbon atoms;

a ureido radical —NH—CO—NH—R′″, wherein R′″ is a unit chosen from a hydrogen atom and linear, branched and cyclic, saturated and unsaturated hydrocarbon-based radicals comprising from 1 to 36 carbon atoms;

an alkylurethane radical of formula —O—CO—NHR″″, wherein R″″ is a unit chosen from linear, branched and cyclic, saturated and unsaturated hydrocarbon-based radicals comprising from 1 to 36 carbon atoms;

a dialkylsiloxane radical of formula:

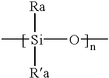

in which:

n is an integer ranging from 1 to 12;

Ra and R′a, which may be identical or different, are each a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, the unit may optionally be substituted with at least one substituent, for example, chosen from the list below;

a trialkylsilane radical of formula:

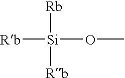

in which Rb, R′b and R″b, which may be identical or different, are each a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, may optionally be substituted with at least one substituent, for example, chosen from the list below; and

a unit chosen from linear, branched and cyclic, saturated and unsaturated hydrocarbon-based radicals comprising from 1 to 36 carbon atoms, the unit may optionally comprise from 1 to 12 hetero atoms chosen from O, N, S and Si and, wherein the unit may optionally be substituted with at least one substituent, for example, chosen from the list below;

wherein at least one of the pair of radicals R1 with R2 and R3 with R4, together with the carbon atoms to which they are attached, may optionally form a ring chosen from a saturated ring and an unsaturated ring, the ring comprising from 5 to 6 carbon atoms in total, wherein the ring may optionally comprise at least one hetero atom chosen from O, N and S, and wherein the ring may optionally be substituted with at least one substituent, for example, chosen from the list below;

wherein the radicals R2 and R3, together with the carbon atoms to which they are attached, may optionally form a ring chosen from a saturated ring and an unsaturated ring, the ring comprising from 5 to 6 carbon atoms in total, wherein the ring may optionally comprise at least one hetero atom chosen from O, N and S, and wherein the ring may optionally be substituted with at least one substituent, for example, chosen from the list below.

Among the at least one substituent which may be borne by the above compounds, such as by at least the hydrocarbon-based radicals of the at least one compound of formula (I), the alkyl groups of the trialkylsilane radical, and the alkyl groups of the dialkylsiloxane radical, mention may be made of the halogens, hydroxyl radicals, amino radicals, nitrile radicals, dialkylsiloxane radicals, and trialkylsilane radicals as defined above; and a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, wherein the unit may optionally be substituted by at least one substituent.

Among the C1-C36 hydrocarbon-based radicals, that may optionally comprise from 1 to 12 hetero atoms chosen from O, N, S and Si, and may be optionally substituted, mention may be made, for example, of saturated and unsaturated C1-C36 alkyl radicals, alkoxy radicals RO— with R=C1-C36, acyloxy radicals R—CO—O— with R=C1-C36 and acyl radicals R—CO— with R=C1-C36.

For example, the at least one ingredient used in the context of the invention may correspond to formula (I) in which:

the radicals R1, R2, R3 and R4, which may be identical or different, are each a unit chosen from a hydrogen atom and linear, branched and cyclic, saturated and unsaturated hydrocarbon-based radicals comprising from 1 to 36 carbon atoms, wherein the unit may optionally comprise from 1 to 8 hetero atoms chosen from O and N, and wherein the unit may optionally be substituted by at least one substituent;

wherein at least one of the pair of radicals R1 with R2 and R3 with R4, together with the carbon atoms to which they are attached, may optionally form a ring chosen from a saturated ring and an unsaturated ring, the ring comprising 6 carbon atoms in total, wherein the ring may optionally comprise at least one hetero atom and may optionally be substituted by at least one substituent, for example, with linear, branched and cyclic, saturated and unsaturated hydrocarbon-based radicals comprising from 1 to 36 carbon atoms, wherein the hydrocarbon-based radicals may optionally comprise from 1 to 8 hetero atoms chosen from O and N, and wherein the hydrocarbon-based radicals may optionally be substituted by at least one substituent, for example, with linear, branched and cyclic, saturated and unsaturated hydrocarbon-based radicals comprising from 1 to 36 carbon atoms; and

wherein the radicals R2 and R3, together with the carbon atoms to which they are attached, may optionally form a ring chosen from a saturated ring and an unsaturated ring, wherein the ring comprises 6 carbon atoms in total, the ring may optionally comprise at least one hetero atom, and the ring may optionally be substituted by at least one substituent, for example, with linear, branched and cyclic, saturated and unsaturated hydrocarbon-based radicals comprising from 1 to 36 carbon atoms, the hydrocarbon-based radicals may optionally comprise from 1 to 8 hetero atoms chosen from O and N, and may optionally be substituted by at least one substituent, for example, with linear, branched and cyclic, saturated and unsaturated hydrocarbon-based radicals comprising from 1 to 36 carbon atoms.

For example, the at least one ingredient chosen from compounds of formula (I) and salts thereof, may be chosen from the compounds corresponding to one of the five formulae below, and salts thereof:

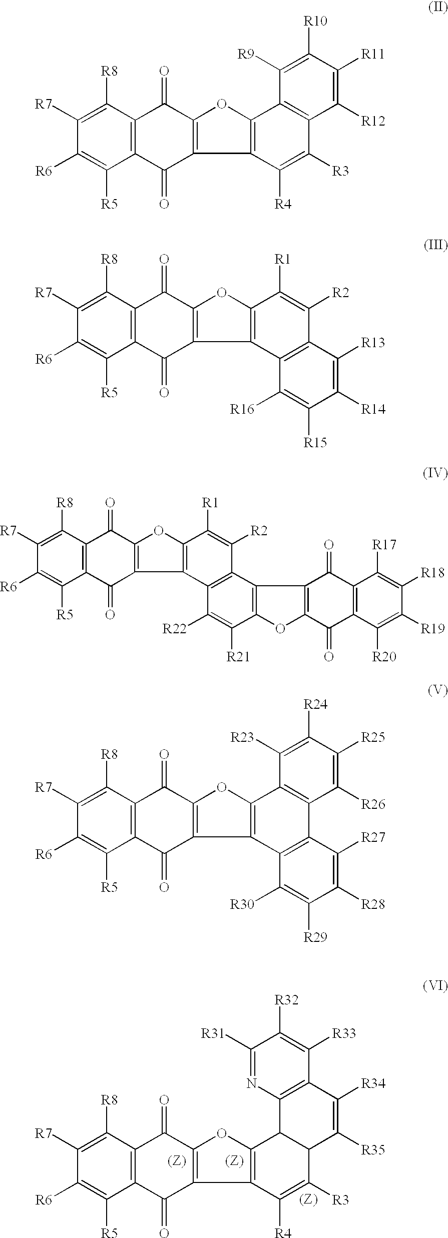

in which:

the radicals R1 to R8, which may be identical or different, are as defined above; and

the radicals R9 to R35, which may be identical or different, have the meanings given for the radicals R1 to R8 above, i.e., chosen from a hydrogen atom; a halogen atom; a hydroxyl radical; an amino radical; a nitro radical; an alkylamido radical; an ureido radical; an alkylurethane radical; a dialkylsiloxane radical; a trialkylsilane radical; a unit chosen from linear, branched and cyclic, saturated and unsaturated hydrocarbon-based radicals comprising from 1 to 36 carbon atoms, wherein the unit may optionally comprise at least one hetero atom chosen from O, N, S and Si and wherein the unit may optionally be substituted with at least one substituent;

it being understood that two adjacent radicals, together with the carbon atoms to which they are attached, may optionally form a ring chosen from a saturated ring and an unsaturated ring, wherein the ring comprises from 5 to 6 carbon atoms in total, wherein the ring may optionally comprise at least one hetero atom chosen from O, N, S, and Si, and wherein the ring may optionally be substituted with at least one substituent, for example, chosen from the above list of substituents.

For example, the at least one ingredient of the invention chosen from compounds of formula (I) and salts thereof, may be chosen from those of formulae (II) to (VI) and, salts thereof, and as a further example, from those of formulae (II) and (III), and salts thereof, in which the various radicals, which may be identical or different, are chosen from:

a hydrogen atom;

a halogen atom, such as chlorine, bromine, iodine and fluorine;

a hydroxyl radical (—OH);

an alkoxy radical (RO—) wherein R is a unit chosen from saturated and unsaturated, linear and branched (C1-C12) alkyl radicals;

a unit chosen from linear and branched, saturated and unsaturated (C1-C12) alkyl radicals;

an acyl radical (R—CO—) wherein R is a unit chosen from saturated and unsaturated, linear and branched (C1-C12) alkyl radicals;

an amino radical —NRR′, wherein R and R′, which may be identical or different, are chosen from a hydrogen atom and linear and branched, saturated and unsaturated hydrocarbon-based radicals comprising from 1 to 12 carbon atoms; and

a nitro radical (—NO₂).

As a further example, the at least one ingredient is chosen from compounds of formula (I) and salts thereof, such as formulae (II) to (VI), and salts thereof, and as a further example, from the compounds of formulae (II) and (III), and salts thereof, in which the various radicals, which may be identical or different, are chosen from:

a hydrogen atom;

a chlorine or bromine atom;

a hydroxyl radical —OH;

an alkoxy radical RO— wherein R is a unit chosen from saturated and unsaturated, linear and branched C1-C6 alkyl radicals, such as, for example, chosen from a methoxy radical, an ethoxy radical, and a propoxy radical; and

a unit chosen from linear and branched, saturated and unsaturated C1-C6 alkyl radicals.

Among the compounds of the invention, mention may be made, for example, of:

among the at least one compound corresponding to formula (II):

dinaphtho(1,2-b:2′,3′-d)furan-7,12-dione

2-hydroxydinaphtho(1,2-b:2′,3′-d)furan-7,12-dione

3-hydroxydinaphtho(1,2-b:2′,3′-d)furan-7,12-dione

4-hydroxydinaphtho(1,2-b:2′,3′-d)furan-7,12-dione

5-methoxydinaphtho(1,2-b:2′,3′-d)furan-7,12-dione

5-chlorodinaphtho(1,2-b:2′,3′-d)furan-7,12-dione

5-ethoxydinaphtho(1,2-b:2′,3′-d)furan-7,12-dione

5-isopropyloxydinaphtho(1,2-b:2′,3′-d)furan-7,12-dione

5-hexyloxydinaphtho(1,2-b:2′,3′-d)furan-7,12-dione

5-(cholest-5-en-3β-ol)dinaphtho(1,2-b:2′,3′-d)furan-7,12-dione

among the at least one compound corresponding to formula (III):

dinaphtho(2,1-b:2′,3′-d)furan-8,13-dione

2-methoxydinaphtho(2,1-b:2′,3′-d)furan-8,13-dione

3-bromodinaphtho(2,1-b:2′,3′-d)furan-8,13-dione

3-hydroxydinaphtho(2,1-b:2′,3′-d)furan-8,13-dione

3-methoxydinaphtho(2,1-b:2′,3′-d)furan-8,13-dione

5-hydroxydinaphtho(2,1-b:2′,3′-d)furan-8,13-dione

among the at least one compound corresponding to formula (IV):

(dinaphtho(2,1-b:2′,3′-d)furan-8,13-dione)-(3,4-b)naphtho(2′,3′-d)furan-5,14-dione

among the at least one compound corresponding to formula (V):

naphtho(2,3-b)phenanthro(9,10-d)furan-10,15-dione

among the at least one compound corresponding to formula (VI):

naphtho(2,3-b)-5-azophenanthro(3′,4′-d)furan-10,15-dione.

Some of these compounds are novel in themselves and therefore form another subject of the invention.

The novel compounds are chosen from one of the formulae (IIa) to (VIa) below, and salts thereof,:

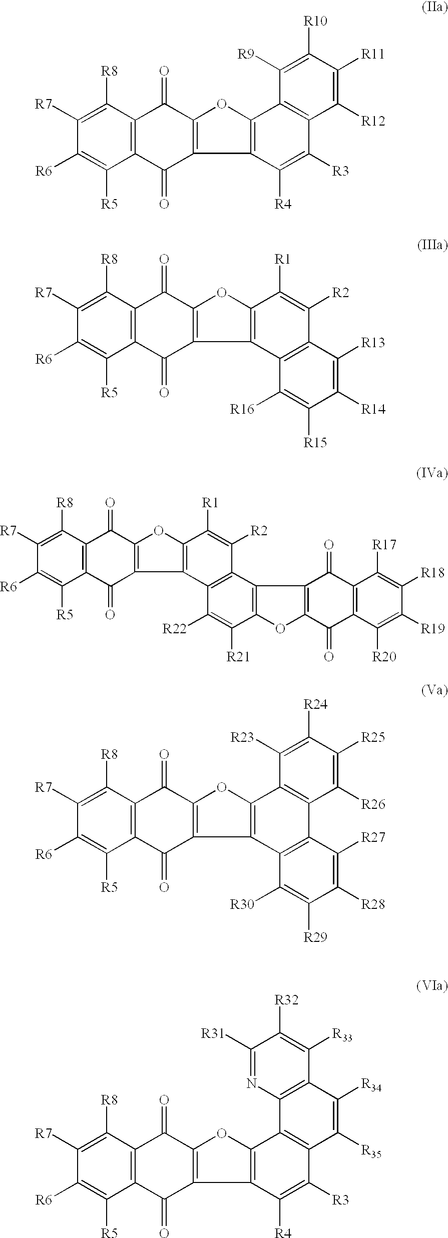

in which:

the radicals R1 to R8, which may be identical or different, are as defined above, and the radicals R9 to R35, which may be identical or different, and have the meanings given for radicals R1 to R8 above, i.e., chosen from a hydrogen atom; a halogen atom; a hydroxyl radical; an amino radical; a nitro radical; an alkylamido radical; an ureido radical; an alkylurethane radical; a dialkylsiloxane radical; a trialkylsilane radical; and a unit chosen from linear, branched and cyclic, saturated and unsaturated hydrocarbon-based radicals comprising from 1 to 36 carbon atoms, wherein the unit may optionally comprise at least one hetero atom chosen from O, N, S and Si and, the unit may optionally be substituted with at least one substituent;

it being understood that two adjacent radicals, together with the carbon atoms to which they are attached, may optionally form a ring chosen from a saturated ring and an unsaturated ring, wherein the ring comprises from 5 to 6 carbon atoms in total, wherein the ring may optionally comprise at least one hetero atom chosen from O, N, S, and Si, and wherein the ring may optionally be substituted with at least one substituent, for example, chosen from the above list;

with the exception of the following compounds:

wherein the radicals R9 to R35, which may be identical or different, and are chosen from the meanings given for the radicals R1 to R8 as defined above, with the exception of the following compounds:

of formula (IIa) or (IIIa) or (Va) in which all the radicals are H;

of formula (IIa) in which R3=OH and all the other radicals are H;

of formula (IIa) in which R3=OCH₃ and all the other radicals are H;

of formula (IIa) in which R10=OH and all the other radicals are H;

of formula (IIa) in which R11=OH and all the other radicals are H;

of formula (IIIa) in which all the radicals are H;

of formula (IIIa) in which R13=OCH₃ and all the other radicals are H;

of formula (IIIa) in which R13=OH and all the other radicals are H;

of formula (IIIa) in which R1=OH and all the other radicals are H;

of formula (IIIa) in which R2=OH and all the other radicals are H;

of formula (IIIa) in which R1=OCH₃ and all the other radicals are H;

of formula (IIa) or (IIIa) in which R5=NO₂ and all the other radicals are H;

of formula (IIa) or (IIIa) in which R8=NO₂ and all the other radicals are H;

of formula (IIa) or (IIIa) in which R5=NH₂ and all the other radicals are H;

of formula (IIa) or (IIIa) in which R8=NH₂ and all the other radicals are H;

of formula (IIa) or (IIIa) in which R5 is benzamido and all the other radicals are H;

of formula (IIa) or (IIIa) in which R8 is benzamido and all the other radicals are H;

of formula (IIIa) in which R8=Br and all the other radicals are H;

of formula (IIIa) in which R8=p-tolylsulphon-amido and all the other radicals are H;

of formula (IIIa) in which R1=OCH₃ and all the other radicals are H;

of formula (IIIa) in which R2=O(CH₂)₂—N(CH₃)₂H.Cl and all the other radicals are H;

of formula (IIIa) in which R2=O(CH₂)₃—N(CH₃)₂H.Cl and all the other radicals are H;

of formula (IIIa) in which R1 is chosen from —CONH-(2′-pyridyl), —CONH-(2′-pyrimidinyl), —CONH-(2′-thiazolyl), —CONH-(3′(H-1,2,4-triazolyl)) and —CONH-phenyl and all the other radicals are H;

of formula (IIIa) in which R1 is chosen from —CONH-(2′-methylphenyl), —CONH-(4′-cyanophenyl), —CONH-(2′(3′-methoxypyridyl)), and —CONH-(4′-methoxyphenyl), R14 and R15 together are —CH═CH—CH═CH— and all the other radicals are H;

of formula (IIIa) in which R1 has the following formula:

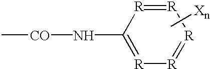

in which R is chosen from N and CH, wherein from 1 to 3 radicals R are N; X is chosen from H, CH₃, C₂H₅, NO₂, OCH₃, CN, SO₂NH₂, CO₂CH₃, CO₂C₂H₅, SO₂NHC₆H₅, Cl, F, Br and I;

and n is a positive integer ranging from 1 to 4.

The at least one ingredient chosen from compounds of formula (I) and salts thereof may be prepared by a person skilled in the art on the basis of his general knowledge.

For example, the at least one ingredient may be prepared from 1-naphthols in the manner described in the publication by Shand et al., Tetrahedron, 1963, vol. 19, pp. 1919-1937, the disclosure of which is incorporated by reference, describing the reaction of 2,3-dichloro-1,4-naphthoquinone with 4-methoxy-1-naphthol in reflux in pyridine.

The at least one ingredient chosen from compounds of formula (I) and salt thereof may be used, in particular as colouring agents, in a composition, such as a cosmetic composition, which may be in the form of a product to be applied to at least one of mucous membranes, semi-mucous membranes, and keratin tissues, such as the skin and superficial body growths (nails, eyelashes, eyebrows, body hair and head hair).

The at least one ingredient of the invention chosen from compounds of formula (I) and salts thereof may be incorporated into the composition, such as a cosmetic composition, in an amount which may be readily determined by a person skilled in the art on the basis of his general knowledge, and which may range generally from 0.01% to 50% by weight relative to the total weight of the composition, for example, in an amount ranging from 0.1% to 20% by weight, and as a further example, from 0.5% to 10% by weight.

Specifically, it has been found that the at least one ingredient according to the invention has at least one good colouring property, such as good colouring strength and photochemical stability, as well as little bleeding in water. Moreover, the at least one ingredient may also have at least one of good covering power and good heat stability.

The term “colouring strength” means the ability of a compound to dye a white mass dispersed in an oily medium. The colouring strength is generally measured using a colorimeter and is expressed by the difference in colour between the pure compound and the dispersed compound. The at least one ingredient or salt thereof according to the invention has a colouring strength which is comparable with or even greater than that of the pigments of the same shade usually used in cosmetics.

The term “bleeding” means the property which a compound has to become dissolved in the medium in which it is dispersed, such that it colours it. This may be quantified by measuring the optical density of the supernatant saturated with colouring agent.

The at least one ingredient according to the invention or salt thereof can have a bleeding which is comparable with or even greater than that of the pigments of the same shade usually used in cosmetics.

The term “covering power” means the ability of a compound dispersed in an oily medium to mask a black and white checkerboard contrast plate. Some of the at least one ingredient and salt thereof according to the invention have good covering power.

Moreover, the at least one ingredient and salt thereof according to the invention can have good heat stability. Thus, they are generally stable after 24 hours at 90° C. and after 2 months at 60° C.

The at least one ingredient and salt thereof can also have good photochemical stability. The expression “photochemical stability” means the ability of a compound not to become coloured by UV irradiation. The photostability is quantified by determining the colorimetric variation between the compacted pure compound before and after UV irradiation. The at least one ingredient and salt thereof according to the invention can have a photochemical stability which is comparable with or even greater than that of the reference pigments in the shade usually used in cosmetics.

The measuring methods are given before the examples.

The composition in accord with the invention contains a cosmetically acceptable medium, i.e., a medium which is compatible with all keratin materials, such as the skin, the nails, the hair, the eyelashes and the eyebrows, mucous membranes and semi-mucous membranes, and any other area of facial and body skin.

The medium may comprise or be in the form of, for example, a suspension, a dispersion, a solution in solvent medium which is optionally thickened, a solution in aqueous-alcoholic medium which is optionally thickened, and a gel; an oil-in-water emulsion; a water-in-oil emulsion; a multiple emulsion; a gel; a mousse; an emulsified gel; a dispersion of vesicles, such as lipid vesicles; a two-phase lotion; a multiphase lotion; a spray; a free powder; a compact powder; a cast powder; or an anhydrous paste. A person skilled in the art may select the appropriate presentation form, as well as the method for preparing it, on the basis of his general knowledge, taking into account on the one hand the nature of the constituents used, such as their solubility in the support, and on the other hand the application envisaged for the composition.

When the composition is in aqueous form, such as in the form of a dispersion, an emulsion or an aqueous solution, it may comprise an aqueous phase, which may comprise at least one of water, a floral water, and a mineral water.

The aqueous phase may also comprise at least one ingredient chosen from alcohols, such as C₁-C₆monoalcohols, and polyols, such as glycerol, butylene glycol, isoprene glycol, propylene glycol, and polyethylene glycol.

When the composition according to the invention is in the form of an emulsion, it may also optionally comprise at least one surfactant, preferably in an amount ranging from 0.01% to 30% by weight relative to the total weight of the composition. The composition according to the invention may also comprise at least one co-emulsifier which may be chosen from oxyethylenated sorbitan monostearate, fatty alcohols, such as stearyl alcohol and cetyl alcohol, and fatty acid esters of polyols, such as glyceryl stearate.

The composition according to the invention may also comprise at least one thickener in concentrations ranging from 0% to 6% by weight relative to the total weight of the composition, chosen from:

polysaccharide biopolymers, such as xanthan gum, carob gum, guar gum, alginates, modified celluloses, starch derivatives, cellulose ether derivatives containing quaternary ammonium groups, and cationic polysaccharides;

synthetic polymers, such as polyacrylic acids, polyvinylpyrrolidone, polyvinyl alcohol and polyacrylamide-based polymers; and

magnesium aluminium silicate.

Depending on the application envisaged, the composition may also comprise at least one film-forming polymer. This is especially the case when it is desired to prepare a composition such as a nail varnish, mascara, eyeliner or hair composition, such as a lacquer. The at least one polymer may be dissolved or dispersed in the cosmetically acceptable medium. For example, the at least one polymer may be present in the form of a solution in an organic solvent or in the form of an aqueous dispersion of film-forming polymer particles. The at least one polymer may be chosen from at least one of nitrocellulose, cellulose acetobutyrate, polyvinyl butyrals, alkyd resins, polyesters, acrylics, vinyls, and polyurethanes.

The composition may also comprise at least one plasticizer, which may be present in an amount ranging from 1% to 40% by weight relative to the total weight of the composition.

The composition according to the invention may also comprise a fatty phase, for example, comprising fatty substances that are liquid at 25° C., such as oils of animal, plant, mineral and synthetic origin; fatty substances that are solid at 25° C., such as waxes of animal, plant, mineral and synthetic origin; pasty fatty substances; gums; and mixtures thereof.

The compositions according to the invention may thus comprise volatile oils, which will evaporate on contact with the skin, but whose presence in the cosmetic composition is useful since they make the composition spread more easily when it is applied to the skin. Such spreading agents referred to herein as “volatile oils” are generally oils having, at 25° C., a saturating vapour pressure at least equal to 0.5 millibar (i.e. 50 Pa).

Mention may thus be made of volatile silicone oils such as:

cyclic volatile silicones comprising from 3 to 8, such as from 4 to 6 silicone atoms,

cyclocopolymers of the dimethylsiloxane/methyl-alkylsiloxane type, and

linear volatile silicones comprising from 2 to 9 silicone atoms.

Mention may also be made of hydrocarbon-based volatile oils, such as isoparaffins, such as isododecane, and fluoro oils.

It is also possible to use non-volatile oils, among which mention may be made of:

poly(C₁-C₂₀)alkylsiloxanes, such as those containing trimethylsilyl end groups, among which mention may be made of linear polydimethylsiloxanes and alkylmethylpolysiloxanes such as cetyldimethicone (CTFA name),

silicones modified with at least one of aliphatic groups optionally fluorinated, aromatic groups optionally fluorinated, and functional groups such as hydroxyl, thiol and amine groups,

phenylsilicone oils, such as those of formula:

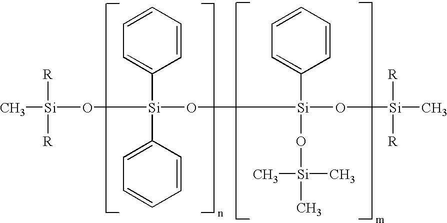

in which R is independently chosen from a (C1-C30) alkyl radical, an aryl radical and an aralkyl radical, n is an integer ranging from 0 to 100, and m is an integer ranging from 0 to 100, with the proviso that the sum of n and m ranges from 1 to 100,

oils of animal, plant or mineral origin, such as animal or plant oils formed by fatty acid esters of polyols, such as liquid triglycerides, for example sunflower oil, corn oil, soybean oil, marrow oil, grape pip oil, sesame oil, hazelnut oil, apricot oil, almond oil and avocado oil; fish oils, glyceryl tricaprocaprylate, and plant and animal oils of formula R1COOR2 in which R1 is a higher fatty acid residue containing from 7 to 19 carbon atoms and R2 is a branched hydrocarbon-based chain containing from 3 to 20 carbon atoms, for example purcellin oil; liquid paraffin, liquid petroleum jelly, perhydrosqualene, wheatgerm oil, beauty-leaf oil, sesame oil, macadamia oil, grape pip oil, rapeseed oil, coconut oil, groundnut oil, palm oil, castor oil, jojoba oil, olive oil or cereal germ oil; fatty acid esters; alcohols; acetylglycerides; alkyl octanoates, polyalkyl octanoates, decanoates and ricinoleates; fatty acid triglycerides; glycerides; and

fluoro oils and perfluoro oils.

The composition according to the invention may also comprise other fatty substances, which may be chosen by a person skilled in the art on the basis of his general knowledge, so as to give the final composition the desired properties, for example in terms of at least consistency and texture. These additional fatty substances may at least be waxes, gums, and pasty fatty substances, of animal, plant, mineral and synthetic origin, as well as mixtures thereof. Mention may be made, for example, of silicone gums; waxes of animal, plant, mineral and synthetic origin such as microcrystalline waxes, paraffin, petrolatum, petroleum jelly, ozokerite wax, montan wax; beeswax, lanolin and its derivatives; candellila wax, ouricury wax, carnauba wax, Japan wax, cocoa butter, cork fibre wax, and sugar cane wax; hydrogenated oils that are solid at 25° C., ozokerites, fatty esters and glycerides that are solid at 25° C.; polyethylene waxes and the waxes obtained by Fischer-Tropsch synthesis; hydrogenated oils that are solid at 25° C.; lanolins; fatty esters that are solid at 25° C.; silicone waxes; and fluoro waxes.

The composition may also contain a particulate phase, which may comprise at least one of pigments, nacres, and fillers usually used in cosmetic compositions. The pigments may be present in the composition in an amount ranging from 0% to 15% by weight relative to the total weight of the final composition, such as in an amount ranging from 8% to 10% by weight, and may be chosen from white and coloured, mineral and organic, and usual and nanometric sizes. Mention may be made of titanium dioxide, zirconium dioxide, cerium dioxide, as well as zinc oxide, iron oxide, chromium oxide, ferric blue, chromium hydrate, carbon black, ultramarines (aluminosilicate polysulphides), manganese pyrophosphate and certain metal powders, such as those of silver or aluminium. Mention may also be made of the lakes commonly used to give the lips and the skin a make-up effect, namely calcium, barium, aluminium salts, zirconium salts, and acidic dyes.

The nacres may be present in the composition in an amount ranging from 0% to 20% by weight relative to the total weight of the composition, such as from 8% to 15% by weight, and may be chosen from natural mother-of-pearl, mica coated with titanium oxide, mica coated with iron oxide, mica coated with natural pigment, and mica coated with bismuth oxy-chloride, as well as coloured titanium mica.

The fillers, which may be present in the composition in an amount ranging from 0% to 30% by weight relative to the total weight of the composition, such as from 5% to 15% by weight, in the composition may be chosen from mineral and synthetic, and lamellar and non-lamellar fillers. Mention may be made of talc, mica, silica, kaolin, Nylon powder, polyethylene powder, TEFLON®, starch, boron nitride, polymer microspheres, such as EXPANCEL (Nobel Industrie), POLYTRAP (Dow Corning) and silicone resin microbeads (for example TOSPEARLS from Toshiba), precipitated calcium carbonate, magnesium carbonate, hydrocarbonate, and metal soaps derived from organic carboxylic acids comprising from 8 to 22 carbon atoms.

The composition may also comprise at least one of a water-soluble dye and a liposoluble dye, such as a natural organic dye, such as cochineal carmine, and a synthetic dye, such as halo acid dyes, azo dyes and anthraquinone dyes. Mention may also be made of mineral dyes such as copper sulphate.

The composition may also comprise at least one additive usually used in the cosmetic field, such as antioxidants, fragrances, essential oils, preserving agents, lipophilic cosmetic active agents, hydrophilic cosmetic active agents, moisturizers, vitamins, essentially fatty acids, sphingolipids, self-tanning agents such as DHA, sunscreens, antifoaming agents, sequestering agents and antioxidants. Needless to say, a person skilled in the art will take care to select the optional additional compounds and the amount thereof, such that the advantageous properties of the composition according to the invention are not, or are not substantially, adversely affected by the addition envisaged.

The cosmetic compositions according to the invention may be, for example,

in the form of a make-up product for at least one of the skin of the face, the body, the lips, and keratin fibres (nails, eyelashes, eyebrows or hair), such as a foundation, a tinted cream, a face powder, an eyeshadow, a free powder, a compact powder, a concealer stick, a cover stick, an eyeliner, a mascara, a lipstick, a nail varnish and a make-up composition for the hair;

in the form of a care product for facial or body skin including the scalp, such as a care composition (day, night, anti-wrinkle, moisturizing, etc.) for the face; a matte-effect composition for the face;

in the form of an antisun composition or artificial tanning (self-tanning) composition; or

in the form of a hair composition, such as a styling cream or gel, an oxidation dye composition or direct dye composition, optionally in the form of a colouring shampoo.

The invention is illustrated in greater detail in the examples which follow.

Measuring Methods

The colorimeter used is a Minolta CR-300 colorimeter.

1/Colouring Strength: Ability to Dye a White Mass (TiO₂) Dispersed in an Oily Medium

Measurement on the pure compound: the pure compound was compacted at 100 bar, in an FAP crucible.

Measurement of the dispersed compound: a mixture was prepared comprising:

| | |
|---|---|
| petroleum jelly | 10 g |
| titanium oxide (untreated) | 4 g |
| test compound | 2 g |

The mixture was homogenized with a spatula. It was ground three times using a three-roll mill (at spacing setting 1).

For the measurement, the mixture was crushed under a glass slide, of the thickness of a microscope slide.

The values of L, a and b were determined using a colorimeter, for the compacted pure compound and for the mixture (petroleum jelly+titanium oxide+compound).

In the L, a and b system, L represents the luminance, a represents the red-green axis (−a=green, +a=red) and b represents the yellow-blue axis (−b=blue, +b=yellow).

The value C which indicates the saturation of the compound was also determined.

2/Covering Power: Ability to Mask a Checkerboard Contrast Plate

An oil-in-water emulsion was prepared comprising:

| | | |
|---|---|---|
| Parleam oil | 22 | g |
| Stearic acid | 1.5 | g |
| Tween 60 (ICl) | 0.9 | g |
| Sipol C16 (Henkel) | 0.5 | g |
| Simulsol 165 (SEPPIC) | 2.1 | g |
| Triethanolamine | 0.75 | g |
| Test compound | 10 | g |
| Propylene glycol | 3 | g |
| Cyclopentadimethylsiloxane | 3 | g |
| Carbopol 981 | 0.15 | g |
| Rhodicare (Rhodia) | 0.2 | g |
| Water | qs 100 | g |

The mixture was ground three times in a three-roll mill.

The emulsion was applied to the contrast leaves (checkerboard with alternate black and white squares); this was done using a spreader, applying a uniform force (90 μm) along the entire card.

The values of (L, a and b) were determined using a colorimeter, on a white background and on a black background.

ΔE^{*} was calculated from the variations ΔL, Δa and Δb according to the following formula:

ΔE*=\sqrt{(ΔL)²+(Δa)²+(Δb)²}

The covering power ΔE^{*} corresponds to the colorimetric difference between the measurement on a white background and that on a black background. When ΔE^{*}<5, the product was considered as having very high covering power; when ΔE^{*}>20, the product was considered as having poor covering power.

3/Bleeding: Ability to Dissolve in a Dispersion Medium

A dispersion was prepared comprising 1% by weight of test compound in an oil (caprylic/capric acid triglycerides).

A dispersion was also prepared comprising 1% by weight of test compound in a mixture of water and 1% by weight of wetting agent (Polysorbate 20).

The mixture was stirred for 10 minutes using a magnetic bar. 10 g of dispersion were weighed out and placed in centrifuge tubes. This material was centrifuged for 30 minutes at 3000 rpm.

The supernatant liquid was taken up and the optical density was determined by measuring the absorbence on a spectrophotometer from 400 to 800 nm, at a speed of 240 nm/min.

The compound was considered to bleed when the optical density was greater than 1.5.

4/Photochemical Stability

The UV stability was carried out by leaving the compacted pure test compound (100 bar, FAP crucible) in the SUNTEST CPS for one hour. The SUNTEST CPS is an apparatus sold by Heraeus. The apparatus had a xenon lamp, which emits a light looking like sunlight. The apparatus was used at 765 watts/m² and emits UVA, UVB, and visible light over a range of 300 to 800 nm.

The difference in colour ΔE before and after the SUNTEST was measured by colorimetry.

The compound was considered as UV-unstable when the ΔE was >6.

5/X-Ray Diffraction:

The X-ray diffraction spectrum characterizes the polymorphic form of the compounds.

Siemens D5005 diffractometer; scintillation detector

copper anticathode, voltage 40 KV, current 40 mA

assembly θ-θ

measuring range: 5° to 30° (fixed sample)

incrementation between each measurement: 0.04°

measuring time per increment: 4 seconds

temperature: (20±1° C.)

fixed slits: 1.6 mm

Kβ filter (Ni)

no internal reference

zeroing procedure with the Siemens slits

experimental data processed using the EVA software (v. 5.0)

### EXAMPLE 1

### Preparation of 5-chlorodinaphtho-(1,2-b:2′,3′-d)furan-7,12-dione

Formula:

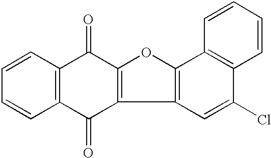

17.86 g (0.1 mol) of 4-chloro-1-naphthol and 22.71 g (0.1 mol) of 2,3-dichloro(1,4)naphthoquinone were dissolved in 300 ml of pyridine at room temperature in a 500 ml three-necked flask. The mixture was maintained at the reflux point of the solvent with stirring for 3 hours. The reaction mixture was cooled to 10° C. over 30 minutes and the solid was filtered off on a sinter funnel. The crystals were washed with water, with ethanol and with isopropyl ether and then dried under vacuum.

30.86 g (yield: 93%) of 5-chlorodinaphtho-(1,2b:2′,3′-d)furan-7,12-dione were obtained in the form of bright yellow crystals.

melting point: 276.4° C. (DSC)

HPTLC: single spot

¹H NMR (CDCl₃): in agreement

elemental analysis:

| | C | H | O | Cl |
|---|---|---|---|---|
| theoretical | 72.20 | 2.73 | 14.43 | 10.65 |
| experimental | 72.40 | 2.67 | 14.71 | 10.74 |

UV-visible spectrum: CHCl₃: λ_{max.} nm (ε): 438.5 (74400); 341.5 (39600); 284.0 (193500); 265.0 (861300); 222.5 (221900); 208.0 (209300)

X-Ray diffraction spectrum

| | | Intensity | |
|---|---|---|---|
| ANGLE 2θ (°) | d (Angstrom) | (count) | Intensity (%) |
| 8.858 | 9.97422 | 1212 | 91.4 |
| 10.545 | 8.38269 | 974 | 73.5 |
| 12.759 | 6.93251 | 1326 | 100 |
| 14.369 | 6.15885 | 366 | 27.6 |
| 15.21 | 5.8203 | 194 | 14.6 |
| 17.439 | 5.08119 | 159 | 12 |
| 17.822 | 4.97271 | 143 | 10.8 |
| 19.769 | 4.48716 | 252 | 19 |
| 21.228 | 4.18188 | 173 | 13 |
| 21.813 | 4.07116 | 126 | 9.5 |
| 22.346 | 3.97513 | 295 | 22.2 |
| 24.67 | 3.60578 | 247 | 18.6 |
| 24.927 | 3.56909 | 209 | 15.8 |
| 25.992 | 3.42522 | 858 | 64.7 |
| 26.525 | 3.3576 | 393 | 29.6 |
| 27.151 | 3.28166 | 692 | 52.2 |
| 28.096 | 3.17331 | 111 | 8.4 |
| 29.412 | 3.03425 | 100 | 7.5 |

### EXAMPLE 2

### Preparation of 5-isopropyloxydinaphtho-(1,2-b:2′,3′-d)furan-7,12-dione

Formula:

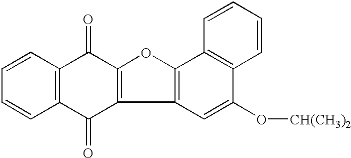

33.1 g (0.146 mol) of 2,3-dichloro(1,4)-naphthoquinone and 29.5 g (0.146 mol) of 4-iso-propoxy-1-naphthol were dissolved in 425 ml of pyridine in a 1-liter three-necked flask. The solution was maintained at 80° C. with stirring for 16 hours. The reaction mixture was cooled to 5° C. and the solid was filtered off on a sinter funnel. The crystals were washed with water and then with methanol and ethanol to give, after drying, 34.4 g (yield: 66%) of 5-isopropyloxy-dinaphtho(1,2-b:2′,3′-d)furan-7,12-dione in the form of bright red crystals.

melting point: 252.9° C. (DSC)

HPTLC: single spot

¹H NMR (CDCl₃): in agreement

elemental analysis:

| | C | H | O |
|---|---|---|---|
| theoretical | 77.52 | 4.53 | 17.96 |
| experimental | 77.52 | 4.47 | 17.90 |

UV-visible spectrum: CHCl₃: λ_{max.} nm (ε): 486.0 (66200), 383.0 (16400), 263.5 (510800), 235.0 (254200), 203.0 (215700).

### EXAMPLE 3

### Preparation of 2-methoxydinaphtho-(2,1-b:2′,3′-d)furan-8,13-dione

Formula:

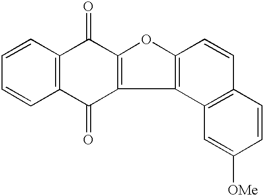

34.05 g (0.15 mol) of 2,3-dichloro(1,4)-naphthoquinone and 26.10 g (0.15 mol) of 7-methoxy-2-naphthol were dissolved, with stirring, in 600 ml of pyridine in a 1-liter three-necked flask and the mixture was heated at 80° C. for 15 hours and then at the reflux point of the solvent for 2 hours. The reaction mixture was cooled to 5° C. and the solid was filtered off and washed with water and then with methanol and with diisopropyl ether and dried under vacuum.

40.27 g (yield: 82%) of 2-methoxydinaphtho-(2,1 b:2′,3′-d)furan-8,13-dione were obtained in the form of orange-red crystals.

melting point: 264.2° C. (DSC)

HPTLC: single spot

¹H NMR (CDCl₃): in agreement

elemental analysis:

| | C | H | O |
|---|---|---|---|
| theoretical | 76.83 | 3.68 | 19.49 |
| experimental | 76.35 | 3.64 | 19.41 |

UV-visible spectrum: CHCl₃: λ_{max.} nm (ε): 467.0 (59700), 322.0 (249900), 285.5 (208000), 246.5 (477600), 222.0 (195500), 205.5 (199800).

X-Ray diffraction spectrum

| | | Intensity | |
|---|---|---|---|
| ANGLE 2θ (°) | d (Angstrom) | (count) | Intensity (%) |
| 7.124 | 12.39813 | 1065 | 69.8 |
| 10.087 | 8.76195 | 1055 | 69.1 |
| 10.694 | 8.26606 | 1526 | 100 |
| 12.37 | 7.14936 | 890 | 58.3 |
| 13.022 | 6.79302 | 273 | 17.9 |
| 14.341 | 6.17099 | 453 | 29.7 |
| 14.784 | 5.98695 | 572 | 37.5 |
| 16.572 | 5.34479 | 214 | 14 |
| 16.696 | 5.30553 | 221 | 14.5 |
| 17.8 | 4.97885 | 198 | 13 |
| 18.612 | 4.76353 | 166 | 10.9 |
| 19.125 | 4.63688 | 304 | 19.9 |
| 19.56 | 4.53465 | 251 | 16.4 |
| 20.39 | 4.35189 | 223 | 14.6 |
| 20.653 | 4.2971 | 309 | 20.2 |
| 22.245 | 3.99296 | 337 | 22.1 |
| 23.524 | 3.77866 | 132 | 8.7 |
| 24.553 | 3.62271 | 168 | 11 |
| 25.317 | 3.51502 | 960 | 56.4 |
| 25.901 | 3.4371 | 1255 | 82.2 |
| 26.2 | 3.39852 | 819 | 53.7 |
| 27.568 | 3.23294 | 393 | 25.8 |
| 27.678 | 3.22028 | 420 | 27.5 |
| 29.622 | 3.01328 | 178 | 11.7 |

### EXAMPLE 4

### Preparation of 3-bromodinaphtho(2,1-b:-2′,3′-d)furan-8,13-dione

Formula:

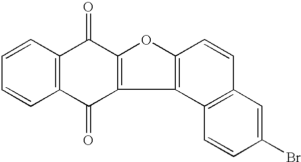

29.23 g (0.13 mol) of 2,3-dichloro(1,4)-naphthoquinone and 28.71 g (0.13 mol) of 6-bromo-2-naphthol were dissolved, with stirring, in 515 ml of pyridine in a 1-liter three-necked flask. The mixture was heated at 80° C. for 18 hours and was then cooled to 5° C. and the solid formed was filtered off. The crystals were washed with water and then with methanol and diisopropyl ether, and were dried under vacuum.

40.35 g (yield: 83%) of 3-bromodinaphtho-(2,1-b:2′,3′-d)furan-8,13-dione were obtained in the form of orange-yellow crystals.

melting point: 329.5° C. (DSC)

HPTLC: single spot

¹H NMR (CDCl₃): in agreement

elemental analysis:

| | C | H | O | Br |
|---|---|---|---|---|
| theoretical | 63.71 | 2.35 | 12.85 | 20.93 |
| experimental | 63.47 | 2.34 | 12.86 | 20.91 |

UV-visible spectrum: CHCl₃: λ_{max.} nm (ε): 443.0 (79500); 295.5 (295600), 284.5 (282000), 254.0 (434700), 242.0 (384900), 229.0 (234500), 208.5 (211100)

### EXAMPLE 5

### Preparation of 3-methoxydinaphtho-(2,1-b:2′,3′-d)furan-8,13-dione

Formula:

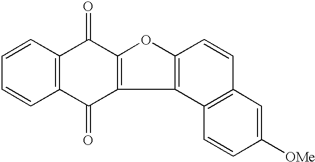

30.19 g (0.133 mol) of 2,3-dichloro(1,4)-naphthoquinone and 23.14 g (0.133 mol) of 6-methoxy-1-naphthol were dissolved in 400 ml of pyridine in a 1-liter three-necked flask. The solution was maintained at 85° C. with stirring for 18 hours. The reaction mixture was cooled to 5° C. and the solid was filtered off on a sinter funnel. The crystals were washed with water and then with methanol and diisopropyl ether and were dried.

24.8 g (yield: 57%) of 3-methoxydinaphtho-(2,1-b:2′,3′-d)furan-8,13-dione were obtained in the form of orange crystals.

melting point: 292.1° C. (DSC)

HPTLC: single spot

¹H NMR (CDCl₃): in agreement

elemental analysis:

| | C | H | O |
|---|---|---|---|
| Theoretical | 76.83 | 3.68 | 19.49 |
| Experimental | 76.37 | 3.60 | 19.34 |

UV-visible spectrum: CHCl₃: λ_{max.} nm (ε): 467.0 (104300), 319.0 (110500), 296.0 (430700), 284.5 (288400), 257.0 (596300), 230.5 (280400).

X-ray diffraction spectrum

| | | Intensity | |
|---|---|---|---|
| ANGLE 2θ (°) | d (Angstrom) | (count) | Intensity (%) |
| 7.02 | 12.5816 | 1800 | 73.2 |
| 10.555 | 8.3741 | 621 | 25.2 |
| 11.834 | 7.47227 | 2460 | 100 |
| 12.213 | 7.24081 | 976 | 39.7 |
| 12.926 | 6.89616 | 1812 | 73.7 |
| 14.147 | 6.2554 | 267 | 10.9 |
| 14.72 | 6.01306 | 201 | 8.2 |
| 15.813 | 5.59984 | 1474 | 59.9 |
| 17.684 | 5.01117 | 223 | 9.1 |
| 18.403 | 4.81697 | 234 | 9.5 |
| 19.778 | 4.4851 | 118 | 4.8 |
| 21.31 | 4.16604 | 145 | 5.9 |
| 23.051 | 3.8552 | 148 | 6 |
| 23.874 | 3.72413 | 840 | 34.1 |
| 24.184 | 3.67713 | 702 | 28.5 |
| 24.963 | 3.56407 | 360 | 14.6 |
| 25.179 | 3.53403 | 395 | 16.1 |
| 25.731 | 3.4594 | 248 | 10.1 |
| 26.28 | 3.38832 | 589 | 23.9 |
| 26.693 | 3.33691 | 731 | 29.7 |
| 27.218 | 3.27369 | 431 | 17.5 |
| 27.872 | 3.19828 | 468 | 19 |
| 28.62 | 3.1164 | 251 | 10.2 |
| 29.469 | 3.02855 | 129 | 5.2 |

### EXAMPLE 6

### Preparation of 5-hexyloxydinaphtho-(1,2-b:2′,3′-d)furan-7,12-dione

Formula:

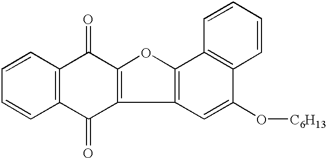

28.4 g (0.116 mol) of 2,3-dichloro(1,4)-naphthoquinone and 26.4 g (0.116 mol) of 4-hexyloxy-1-naphthol were dissolved in 340 ml of pyridine in a 1-liter three-necked flask. The solution was maintained at 75° C. with stirring for 16 hours. The reaction mixture was cooled to 5° C. and the solid was filtered off on a sinter funnel. The crystals were washed with water and then with methanol and ethanol and were dried.

27.0 g (yield: 58%) of 5-hexyloxydinaphtho-(1,2-b:2′,3′-d)furan-7,12-dione were obtained in the form of orange-red crystals.

melting point: 173.9° C. (DSC)

HPTLC: single spot

¹H NMR (CDCl₃): in agreement

elemental analysis:

| | C | H | O |
|---|---|---|---|
| Theoretical | 78.38 | 5.57 | 16.06 |
| Experimental | 78.13 | 5.67 | 16.01 |

UV-visible spectrum: CHCl₃: λ_{max.} nm (ε): 485.5 (50500), 266.5 (378900), 235.5 (198600), 223.0 (197000), 206.0 (182700).

### EXAMPLE 7

### Preparation of 5-(cholest-5-en-3β-ol)-dinaphtho(1,2-b:2′,3′-d)furan-7,12-dione

Formula:

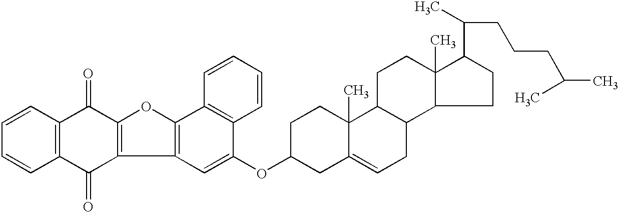

0.602 g (2.65 mmol) of 2,3-dichloro(1,4)-naphthoquinone and 1.40 g (2.65 mmol) of 4-((3β-cholest-5-en-3-oxy)naphthen-1-ol were dissolved in 15 ml of pyridine in a 100 ml three-necked flask. The solution was maintained at 85° C. with stirring for 17 hours. The reaction mixture was cooled to 5° C. and the solid was filtered off on a sinter funnel. The crystals were washed with water and then with methanol and heptane, and were then dried.

0.89 g (yield: 49%) of 5-((3β)-cholest-5-en-3-ol)dinaphto(1,2-b:2′,3′-d)furan-7,12-dione was obtained in the form of bright red crystals.

melting point: 285.6° C. (DSC)

HPTLC: single spot

¹H NMR (CDCl₃): in agreement

mass spectrum: in agreement

### EXAMPLE 8

### Preparation of (dinaphtho(2,1-b:2′,3′-d)-furan-8,13-dione)(3,4-b]naphtho(2′,3′-d)furan-5,14-dione

Formula:

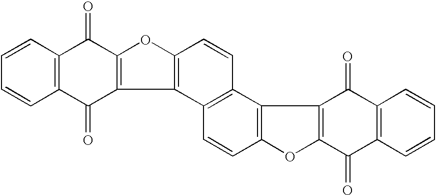

4.54 g (20 mmol) of 2,3-dichloro(1,4)naphthoquinone and 1.60 g (10 mmol) of 2,6-naphthalenediol were dissolved in 55 ml of pyridine in a 100 ml three-necked flask. The solution was maintained at 80° C. with stirring for 18 hours and the reaction mixture was then cooled to 5° C. and the solid was filtered off on a sinter funnel. The crystals were washed with methanol and then with dichloromethane and were dried.

4.66 g (yield: 99%) of the desired compound were obtained in the form of insoluble organge-brown crystals.

HPTLC: single spot

mass spectrum: in agreement

### EXAMPLE 9

### Preparation of naphtho(2,3-b)-5-azaphenanthro(3′,4′-d)furan-10,15-dione

Formula:

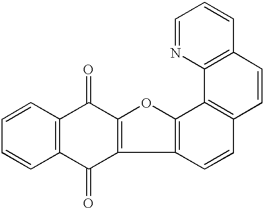

2.27 g (0.010 mol) of 2,3-dichloro(1,4)naphthoquinone and 1.95 g (0.010 mol) of benzo(h)quinolin-10-ol were dissolved in 35 ml of pyridine in a 100 ml three-necked flask. The solution was maintained at 80° C. with stirring for 18 hours. Next, the reaction mixture was then cooled to 0° C. and a solid was extracted with ethyl acetate and purified on a column of silica (eluent: 9 CH₂Cl₂/1 EtOAc). 0.20 g (yield: 5%) of the desired compound was finally isolated in the form of orange crystals.

melting point: greater than 350° C. (DSC)

HPTLC: single spot

¹H NMR (CDCl₃): in agreement

### EXAMPLE 10

The colouring strength, the covering power, the photochemical stability and the bleeding of certain compounds according to the invention were measured.

1/Colouring Strength

The L, a and b values below were obtained:

| Compound | L | a | b | c |
|---|---|---|---|---|
| 5-Chlorodinaphtho(1,2-b:2′,3′-d)furan-7,12- | 85.7 | −2.7 | 103 | 103 |
| dione | | | | |
| 5-Ethoxydinaphtho(1,2-b:2′,3′-d)furan-7,12- | 35.0 | 44.4 | 28.6 | 52.8 |
| dione | | | | |
| 5-Isopropyloxydinaphtho(1,2-b:2′,3′-d)furan- | 44.9 | 54.9 | 47.3 | 72.4 |
| 7,12-dione | | | | |
| Dinaphtho(2,1-b:2′,3′-d)furan-8,13-dione | 75.6 | 10.7 | 88.0 | 88.7 |
| 2-Methoxydinaphtho(2,1-b:2′,3′-d)furan- | 54.5 | 53.0 | 58.4 | 78.8 |
| 8,13-dione | | | | |
| 3-Bromodinaphtho(2,1-b:2′,3′-d)furan-8,13- | 82.5 | 7.6 | 99.1 | 99.4 |
| dione | | | | |
| 3-Methoxydinaphtho(2,1-b:2′,3′-d)furan- | 70.7 | 34.2 | 85.8 | 92.4 |
| 8,13-dione | | | | |
| 5-Hexyloxydinaphtho(1,2-b:2′,3′-d)furan- | 48.9 | 50.8 | 51.0 | 72.0 |
| 7,12-dione | | | | |
| 2-Hydroxydinaphtho(1,2-b:2′,3′-d)furan- | 47.4 | 43.9 | 45.0 | 62.9 |
| 7,12-dione | | | | |
| Dinaphtho(1,2-b:2′,3′-d)furan-7,12-dione | 88.5 | 0.19 | 108 | 108 |
| 5-Hydroxydinaphtho(2,1-b:2′,3′-d)furan- | 22.4 | 16.9 | 9.5 | 19.4 |
| 8,13-dione | | | | |

These compounds thus have pure and saturated colours, as well as a colouring strength which was at least comparable to that of the usual pigments.

2/Covering power

| | |
|---|---|
| For 5-hydroxydinaphtho(2,1-b:2′,3′-d)furan-8,13-dione: | ΔE* = 11.1 |

The compound thus has good covering power.

3/Photochemical stability

| | |
|---|---|
| For 5-Chlorodinaphtho(1,2-b:2′,3′-d)furan-7,12-dione | |
| For 5-Ethoxydinaphtho(1,2-b:2′,3′-d)furan-7,12-dione | ΔE = 1.36 |
| For 5-Isopropyloxydinaphtho(1,2-b:2′,3′-d)furan-7,12-dione | |
| For dinaphtho(2,1-b:2′,3′-d)furan-8,13-dione | ΔE = 0.24 |
| For 2-methoxydinaphtho(2,1-b:2′,3′-d)furan-8,13-dione | |
| For 3-bromodinaphtho(2,1-b:2′,3′-d)furan-8,13-dione | ΔE = 0.81 |
| For 3-methoxydinaphtho(2,1-b:2′,3′-d)furan-8,13-dione | ΔE = 0.23 |
| For 5-hexyloxydinaphtho(1,2-b:2′,3′-d)furan-7,12-dione | |
| For 2-hydroxydinaphtho(1,2-b:2′,3′-d)furan-7,12-dione | ΔE = 0.28 |
| For dinaphtho(1,2-b:2′,3′-d)furan-7,12-dione | |
| For 5-hydroxydinaphtho(2,1-b:2′,3′-d)furan-8,13-dione | ΔE = 2.61 |
| | ΔE = 0.81 |
| | ΔE = 0.91 |
| | ΔE = 1.32 |
| | ΔE = 3.91 |
| | ΔE = 0.10 |

The compounds were thus photochemically stable.

4/Bleeding

| | |
|---|---|
| a/in water | |
| For 5-Chlorodinaphtho(1,2-b:2′,3′-d)furan-7,12-dione | |
| For 5-Ethoxydinaphtho(1,2-b:2′,3′-d)furan-7,12-dione | O.D. = 0.02 |
| For 5-Isopropyloxydinaphtho(1,2-b:2′,3′-d)furan-7,12-dione | |
| For dinaphtho(2,1-b:2′,3′-d)furan-8,13-dione | O.D. = 0.27 |
| For 2-methoxydinaphtho(2,1-b:2′,3′-d)furan-8,13-dione | |
| For 3-bromodinaphtho(2,1-b:2′,3′-d)furan-8,13-dione | O.D. = 0.03 |
| For 3-methoxydinaphtho(2,1-b:2′,3′-d)furan-8,13-dione | O.D. = 0.01 |
| For 5-hexyloxydinaphtho(1,2-b:2′,3′-d)furan-7,12-dione | |
| For 2-hydroxydinaphtho(1,2-b:2′,3′-d)furan-7,12-dione | O.D. = 0.30 |
| For dinaphtho(1,2-b:2′,3′-d)furan-7,12-dione | |
| For 5-hydroxydinaphtho(2,1-b:2′,3′-d)furan-8,13-dione | O.D. = 0.03 |
| | O.D. = 0.04 |
| | O.D. = 0.29 |
| | O.D. = 0.23 |
| | O.D. = 0.13 |
| | O.D. = 0.16 |
| b/in oil (triglycerides) | |
| For 3-bromodinaphtho(2,1-b:2′,3′-d)furan-8,13-dione | |
| For 3-methoxydinaphtho(2,1-b:2′,3′-d)furan-8,13-dione | O.D. = 0.92 |
| | O.D. = 0.88 |

The compounds thus gave little bleeding in water and oil.

### EXAMPLE 11

The colouring strength, the photochemical stability and the bleeding of compounds according to the invention were compared with pigments of the prior art.

1/Colouring strength

| Compound | L | a | b | c |
|---|---|---|---|---|
| 3-Bromodinaphtho(2,1-b:2′,3′-d) | 82.5 | 7.6 | 99.1 | 99.4 |
| furan-8,13-dione | | | | |
| FD&C Yellow No. 5 | 83.5 | 16.1 | 98.8 | 100 |
| 3-Methoxydinaphtho(2,1-b:2′,3′-d) | 70.7 | 34.2 | 85.8 | 92.4 |
| furan-8,13-dione | | | | |
| FD&C Yellow No. 6 | 58.2 | 51.4 | 73.3 | 89.5 |

The compounds according to the invention thus have a colour which was at least as saturated, if not more so, and a colouring strength which was at least comparable to that of a common pigment of the same shade.

2/Photochemical stability

| | |
|---|---|
| For 3-bromodinaphtho(2,1-b:2′,3′-d)furan-8,13-dione | |
| (yellow) | |
| For FD&C Yellow No. 5 (yellow) | ΔE = 2.61 |
| | ΔE = 6.63 |
| For 3-methoxydinaphtho(2,1-b:2′,3′-d)furan-8,13-dione | |
| (orange) | |
| For FD&C Yellow No. 6 (orange) | |
| | ΔE = 0.81 |
| | ΔE = 10.06 |

The compounds according to the invention were thus more photochemically stable than certain common pigments.

3/Bleeding in water

| | |
|---|---|
| For 3-bromodinaphtho(2,1-b:2′,3′-d)furan-8,13-dione | |
| For FD&C Yellow No. 5 (yellow) | O.D. = 0.03 |
| | O.D. = 0.08 |
| For 3-methoxydinaphtho(2,1-b:2′,3′-d)furan-8,13-dione | |
| For FD&C Yellow No. 6 (orange) | |
| | O.D. = 0.04 |
| | O.D. = 0.58 |

The compounds according to the invention thus bled less in water than certain common pigments.

### EXAMPLE 12

A tinted cream of oil-in-water emulsion type was prepared, comprising:

| | | |
|---|---|---|
| parleam oil | 22 | g |
| stearic acid | 1.5 | g |
| Polysorbate 60 (Tween 60 from ICl) | 0.9 | g |
| cetyl alcohol | 0.5 | g |
| glyceryl monostearate/PEG-100 stearate mixture | | |
| triethanolamine | 2.1 | g |
| compound of Example 3 | 0.75 | g |
| propylene glycol | 10 | g |
| cyclopentadimethylsiloxane | 3 | g |
| Carbopol 981 | 3 | g |
| xanthan gum | 0.15 | g |
| water | qs 0.2 | g |
| | 100 | g |

### EXAMPLE 13

An eyeshadow comprising the ingredients below was prepared:

| | | |
|---|---|---|
| talc | 38 | g |
| mica | 20 | g |
| bismuth oxychloride | 8 | g |
| zinc stearate | 3 | g |
| Nylon powder | 20 | g |
| compound of Example 1 | 5 | g |
| fatty binder | qs 100 | g |

### EXAMPLE 14

### Preparation of 3-Hydroxy-dinaphtho(2,1-b;2′,3′-d)furan-8,13-dione

16.02g (0,1 mol) of 7-hydroxy-naphtalen-1-ol and 22,71 g (0.1 mol) of 2,3-dichloro-(1,4)naphtoquinone were dissolved in 300 ml of pyridine at room temperature in a 500 ml three-necked flask. The mixture was maintained at the reflux point of the solvent with stirring for 3 hours. The reaction mixture was cooled to 10° C. over 30 minutes and the solid was filtered off on a sinter funnel. The crystals were washed with water, with ethanol and with isopropyl ether and then dried under vacuum.

19.8 g (yield.:63%) of 3-Hydroxy-dinaphtho(2,1-b;2′,3′-d)furan-8,13-dione were obtained in the form of red crystals.

| | |
|---|---|
| Melting point | 317.6° C. (DSC) |
| HPTLC | single spot |
| ¹H NMR (CDCl₃) | in agreement |

elemental analysis:

| | C | H | O |
|---|---|---|---|
| Theorical | 76.43 | 3.21 | 20.36 |
| Experimental | 75.88 | 3.19 | 20.45 |

X-Ray diffraction Spectrum:

| | | Intensity | |
|---|---|---|---|
| ANGLE 2θ(°) | d (Angstrom) | (count) | Intensity (%) |
| 6.528 | 13.52963 | 243 | 12 |
| 6.952 | 12.70451 | 331 | 16.4 |
| 8.236 | 10.72614 | 2021 | 100 |
| 9.187 | 9.61821 | 573 | 28.4 |
| 9.572 | 9.23249 | 298 | 14.7 |
| 10.906 | 8.10589 | 644 | 31.9 |
| 11.711 | 7.55029 | 278 | 13.8 |
| 12.086 | 7.3167 | 559 | 27.7 |
| 12.53 | 7.05882 | 439 | 21.7 |
| 14.525 | 6.09312 | 266 | 13.2 |
| 15.084 | 5.86885 | 332 | 16.4 |
| 15.676 | 5.64831 | 367 | 18.2 |
| 17.033 | 5.20123 | 362 | 17.9 |
| 17.909 | 4.94866 | 289 | 14.3 |
| 18.687 | 4.74446 | 205 | 10.1 |
| 19.048 | 4.65538 | 255 | 12.6 |
| 20.72 | 4.28332 | 181 | 9 |
| 21.244 | 4.1788 | 236 | 11.7 |
| 21.736 | 4.08539 | 182 | 9 |
| 22.719 | 3.91078 | 351 | 17.4 |
| 23.676 | 3.75484 | 261 | 12.9 |
| 24.306 | 3.65894 | 261 | 12.9 |
| 25.346 | 3.51103 | 541 | 26.8 |
| 25.956 | 3.42994 | 246 | 13.7 |
| 26.799 | 3.32394 | 563 | 27.9 |
| 27.16 | 3.28054 | 405 | 20 |
| 27.881 | 3.19734 | 391 | 19.3 |
| 28.299 | 3.15109 | 375 | 18.6 |
| 29.433 | 3.0322 | 161 | 8 |

### EXAMPLE 15

### Preparation of 2-Hydroxy-dinaphtho(1,2-b:2′,3′-d)furan-7,12-dione

16.02g (0.1 mol) of 7-hydroxy-naphtalen-2-ol and 22,71g (0.1 mol) of 2,3-dichloro-(1,4)naphtoquinone were dissolved in 300 ml of pyridine at room temperature in a 500 ml three-necked flask. The mixture was maintained at the reflux point of the solvent with stirring for 3 hours. The reaction mixture was cooled to 10° C. over 30 minutes and the solid was filtered off on a sinter funnel. The crystals were washed with water, with ethanol and with isopropyl ether and then dried under vacuum.

16.34g (yield.:52%) of 2-Hydroxy-dinaphtho(1,2-b;2′,3′-d)furan-7,12-dione were obtained in the form of red crystals.

| | |
|---|---|
| Melting point: | 332.4° C. (DSC) |
| HPTLC: | Single spot |
| ¹H NMR (CDCl₃) | in agreement |

elemental analysis:

| | C | H | O |
|---|---|---|---|
| Theorical | 76.43 | 3.21 | 20.36 |
| Experimental | 75.57 | 3.20 | 20.45 |

X-Ray Diffraction Spectrum:

| ANGLE 2θ(°) | d (Angstrom) | Intensity (count) | Intensity (%) |
|---|---|---|---|
| 7.227 | 12.22148 | 2302 | 100 |
| 9.337 | 9.46393 | 397 | 17.2 |
| 11.298 | 7.82508 | 330 | 14.3 |
| 12.341 | 7.16606 | 231 | 10 |
| 14.451 | 6.12422 | 1694 | 73.6 |
| 15.747 | 5.62309 | 540 | 23.5 |
| 16.866 | 5.25228 | 279 | 12.1 |
| 17.45 | 5.07802 | 195 | 8.5 |
| 18.023 | 4.91781 | 218 | 9.5 |
| 18.748 | 4.72927 | 165 | 7.2 |
| 19.558 | 4.53501 | 273 | 11.9 |
| 20.862 | 4.25446 | 238 | 10.3 |
| 21.808 | 4.07194 | 152 | 6.6 |
| 22.785 | 3.89965 | 1058 | 46 |
| 23.575 | 3.77064 | 267 | 11.6 |
| 24.725 | 3.59788 | 178 | 7.7 |
| 25.276 | 3.52067 | 281 | 12.2 |
| 26.065 | 3.41582 | 118 | 5.1 |
| 26.873 | 3.31496 | 1287 | 55.9 |
| 28.836 | 3.09358 | 721 | 31.3 |
| 29.343 | 3.04127 | 132 | 5.7 |

What is claimed is:

1. A cosmetic composition comprising at least one ingredient chosen from compounds of formula (I) and salts thereof: 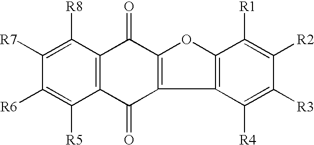
in which the radicals R1 to R8, which may be identical or different, are chosen from:
a hydrogen atom;
a halogen atom;
a hydroxyl radical;
an amino radical —NRR′, wherein R and R′, which may be identical or different, are each a unit chosen from a hydrogen atom and linear, branched and cyclic, saturated and unsaturated hydrocarbon-based radicals comprising from 1 to 36 carbon atoms;
a nitro radical;
an alkylamido radical —NH—CO—R″ wherein R″ is a unit chosen from linear, branched and cyclic, saturated and unsaturated hydrocarbon-based radicals comprising from 1 to 36 carbon atoms;
a ureido radical —NH—CO—NH—R′″ wherein R′″ is a unit chosen from a hydrogen atom and linear, branched and cyclic, saturated and unsaturated hydrocarbon-based radicals comprising from 1 to 36 carbon atoms;
an alkylurethane radical of formula —O—CO—NHR″″ wherein R″″ is a unit chosen from linear, branched and cyclic, saturated and unsaturated hydrocarbon-based radicals comprising from 1 to 36 carbon atoms;
a dialkylsiloxane radical of formula: 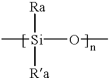
in which
n is an integer ranging from 1 to 12;
Ra and R′a, which may be identical or different, are each a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, wherein the unit may be optionally substituted with at least one substituent;
a trialkylsilane radical of formula: 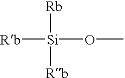
in which Rb, R′b and R″b, which may be identical or different, are each a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, wherein the unit may be optionally substituted with at least one substituent; and
a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, wherein the unit may be optionally substituted with at least one substituent;
wherein at least one of the pair of radicals R1 with R2 and R3 with R4, together with the carbon atoms to which they are attached, may optionally form a ring chosen from a saturated ring and an unsaturated ring, the ring comprising from 5 to 6 carbon atoms in total, wherein the ring may optionally comprise at least one hetero atom chosen from O, N and S, and, wherein the ring may be optionally substituted with at least one substituent;
wherein the radicals R2 and R3, together with the carbon atoms to which they are attached, may optionally form a ring chosen from a saturated ring and an unsaturated ring, the ring comprising from 5 to 6 carbon atoms in total, wherein the ring may optionally comprise at least one hetero atom chosen from O, N and S, and, wherein the ring may be optionally substituted with at least one substituent.

2. The composition according to claim 1, wherein the composition further comprises a cosmetically acceptable medium.

3. The composition according to claim 1, wherein the halogen atom is chosen from chlorine, bromine, iodine, and fluorine.

4. The composition according to claim 1, wherein
the radicals R1, R2, R3 and R4, which may be identical or different, are each a unit chosen from a hydrogen atom and linear, branched and cyclic, saturated and unsaturated hydrocarbon-based radicals comprising from 1 to 36 carbon atoms, wherein the unit may optionally comprise from 1 to 8 hetero atoms chosen from O and N, and wherein the unit may be substituted by at least one substituent;
wherein at least one of the pair of radicals R1 with R2 and R3 with R4, together with the carbon atoms to which they are attached, may optionally form a ring chosen from a saturated ring and an unsaturated ring, the ring comprising 6 carbon atoms in total, wherein the ring may comprise at least one hetero atom and wherein the ring may optionally be substituted with a unit chosen from linear, branched and cyclic, saturated and unsaturated hydrocarbon-based radicals comprising from 1 to 36 carbon atoms, wherein the hydrocarbon-based radicals may optionally comprise from 1 to 8 hetero atoms chosen from O and N, and, wherein the hydrocarbon-based radicals may optionally be substituted with at least one unit chosen from linear, branched and cyclic, saturated and unsaturated hydrocarbon-based radicals comprising from 1 to 36 carbon atoms;
wherein the radicals R2 and R3, together with the carbon atoms to which they are attached, may optionally form a ring chosen from a saturated ring and an unsaturated ring comprising 6 carbon atoms in total, wherein the ring may optionally comprise at least one hetero atom, and wherein the ring may optionally be substituted with a unit chosen from linear, branched and cyclic, saturated and unsaturated hydrocarbon-based radicals comprising from 1 to 36 carbon atoms, wherein the hydrocarbon-based radicals may optionally comprise from 1 to 8 hetero atoms chosen from O and N, and wherein the hydrocarbon-based radicals may optionally be substituted with at least one unit chosen from linear, branched and cyclic, saturated and unsaturated hydrocarbon-based radicals comprising from 1 to 36 carbon atoms.

5. The composition according to claim 1, wherein the compounds of formula (I) and salts thereof correspond to one of the formulae (II) to (VI) below and salts thereof: 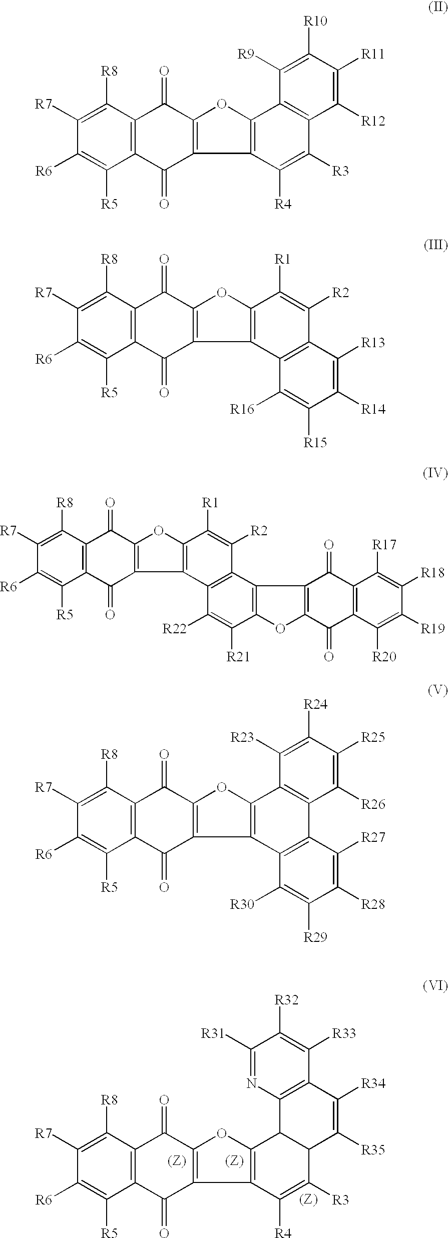
in which:
the radicals R1 to R8, which may be identical or different, are as defined in claim 1, and
the radicals R9 to R35, which may be identical or different, are chosen from the meanings given for the radicals R1 to R8 as defined in claim 1;
it being understood that two adjacent radicals, together with the carbon atoms to which they are attached, may optionally form a ring chosen from a saturated ring and an unsaturated ring, the ring comprising from 5 to 6 carbon atoms in total, wherein the ring may optionally comprise at least one hetero atom chosen from O, N, S and Si and wherein the ring may optionally be substituted with at least one substituent.

6. The composition according to claim 1, wherein the radicals R1 to R8, which may be identical or different, of the compounds of formula (I) and salts thereof are chosen from:
a hydrogen atom;
a halogen atom chosen from chlorine, bromine, iodine and fluorine;
a hydroxyl radical;
an alkoxy radical RO—, wherein R is a unit chosen from saturated and unsaturated, linear and branched C1-C12 alkyl radicals;
a unit chosen from linear and branched, saturated and unsaturated C1-C12 alkyl radicals;
an acyl radical R—CO—, wherein R is unit chosen from saturated and unsaturated, linear and branched C1-C12 alkyl radicals;
an amino radical —NRR; wherein R and R′, which may be identical or different, are chosen from a hydrogen atom and linear and branched, saturated and unsaturated hydrocarbon-based radicals comprising from 1 to 12 carbon atoms; and
a nitro radical.

7. The composition according to claim 5, wherein all of the radicals, which may be identical or different, in each of formulae (II) to (VI) and salts thereof are chosen, from:
a hydrogen atom;
a halogen atom chosen from chlorine, bromine, iodine and fluorine;
a hydroxyl radical;
an alkoxy radical RO—, wherein R is a unit chosen from saturated and unsaturated, linear and branched C1-C12 alkyl radicals;
a unit chosen from saturated and unsaturated, linear and branched C1-C12 alkyl radicals;
an acyl radical R—CO—, wherein R is a unit chosen from saturated and unsaturated, linear and branched C1-C12 alkyl radicals;
an amino radical —NRR; wherein R and R′, which may be independent or different, are chosen from a hydrogen atom and linear and branched, saturated and unsaturated hydrocarbon-based radicals comprising from 1 to 12 carbon atoms; and
a nitro radical.

8. The composition according to claim 5, wherein all of the radicals, which may be independent or different, in each of formulae (II) and (III) and salts thereof are chosen from:
a hydrogen atom;
a halogen atom chosen from chlorine, bromine, iodine and fluorine;
a hydroxyl radical;
an alkoxy radical RO—, wherein R is a unit chosen from saturated and unsaturated, linear and branched C1-C12 alkyl radicals;
a unit chosen from saturated and unsaturated, linear and branched C1-C12 alkyl radicals;
an acyl radical R—CO—, wherein R is a unit chosen from saturated and unsaturated, linear and branched C1-C12 alkyl radicals;
an amino radical —NRR; wherein R and R′, which may be identical or different, are chosen from a hydrogen atom and linear and branched, saturated and unsaturated hydrocarbon-based radicals comprising from 1 to 12 carbon atoms; and
a nitro radical.

9. The composition according to claim 1, wherein the radicals R1 to R8, which may be independent or different, of the compounds of formula (I) and salts thereof are chosen from:
a hydrogen atom;
a chlorine atom or a bromine atom;
a hydroxyl radical;
an alkoxy radical RO— wherein R is a unit chosen from saturated and unsaturated, linear and branched C1-C6 alkyl radicals; and
a unit chosen from linear and branched, saturated and unsaturated C1-C6 alkyl radicals.

10. The composition according to claim 5, wherein all the radicals, which may be identical or different, in each of formulae (II) to (VI) and salts thereof are chosen from:
a hydrogen atom;
a chlorine or bromine atom;
a hydroxyl radical;
an alkoxy radical RO— wherein R is a unit chosen from saturated and unsaturated, linear and branched C1-C6 alkyl radicals; and
a unit chosen from linear and branched, saturated and unsaturated C1-C6 alkyl radicals.

11. The composition according to claim 5, wherein all of the radicals, which may be identical or different, in each of formulae (II) and (III) and salts thereof are chosen from:
a hydrogen atom;
a chlorine or bromine atom;
a hydroxyl radical;
an alkoxy radical RO— wherein R is a unit chosen from linear and branched, saturated and unsaturated C1-C6 alkyl radicals; and
a unit chosen from linear and branched, saturated and unsaturated C1-C6 alkyl radicals.

12. The composition according to claim 9, wherein the R of the alkoxy radical is chosen from a methoxy radical, an ethoxy radical, and a propoxy radical.

13. The composition according to claim 10, wherein the R of the alkoxy radical is chosen from a methoxy radical, an ethoxy radical, and a propoxy radical.

14. The composition according to claim 11, wherein the R of the alkoxy radical is chosen from a methoxy radical, an ethoxy radical, and a propoxy radical.

15. The composition according to claim 1, wherein the compounds of formula (I) are chosen from:
dinaphtho(1,2-b:2′,3′-d)furan-7,12-dione;
2-hydroxydinaphtho(1,2-b:2′,3′-d)furan-7,12-dione;
3-hydroxydinaphtho(1,2-b:2′,3′-d)furan-7,12-dione;
4-hydroxydinaphtho(1,2-b:2′,3′-d)furan-7,12-dione;
5-methoxydinaphtho(1,2-b:2′,3′-d)furan-7,12-dione;
5-chlorodinaphtho(1,2-b:2′,3′-d)furan-7,12-dione;
5-ethoxydinaphtho(1,2-b:2′,3′-d)furan-7,12-dione;
5-isopropyloxydinaphtho(1,2-b:2′,3′-d)furan-7,12-dione;
5-hexyloxydinaphtho(1,2-b:2′,3′-d)furan-7,12-dione;
5-(cholest-5-en-3β-ol)dinaphtho(1,2-b:2′,3′-d)furan-7,12-dione;
dinaphtho(2,1-b:2′,3′-d)furan-8,13-dione;
2-methoxydinaphtho(2,1-b:2′,3′-d)furan-8,13-dione;
3-bromodinaphtho(2,1-b:2′,3′-d)furan-8,13-dione;
3-hydroxydinaphtho(2,1-b:2′,3′-d)furan-8,13-dione;
3-methoxydinaphtho(2,1-b:2′,3′-d)furan-8,13-dione;
5-hydroxydinaphtho(2,1-b:2′,3′-d)furan-8,13-dione;
(dinaphtho(2,1-b:2′,3′-d)furan-8,13-dione)-(3,4-b)naphtho(2′,3′-d)furan-5,14-dione;
naphtho(2,3-b)phenanthro(9,10-d)furan-10,15-dione; and
naphtho(2,3-b)-5-azophenanthro(3′,4′-d)furan-10,15-dione.

16. The composition according to claim 1, wherein the at least one ingredient is present in the composition in an amount ranging from 0.1% to 50% by weight relative to the total weight of the composition.

17. The composition according to claim 16, wherein the at least one ingredient is present in the composition in an amount ranging from 0.1% to 20% by weight relative to the total weight of the composition.

18. The composition according to claim 16, wherein the at least one ingredient is present in the composition in an amount ranging from 0.5% to 10% by weight relative to the total weight of the composition.

19. The composition according to claim 2, wherein the cosmetically acceptable medium comprises a suspension, a dispersion, a solution in solvent medium which is optionally thickened, a solution in aqueous-alcoholic medium which is optionally thickened, a gelled solution; an oil-in-water emulsion; a water-in-oil emulsion; a multiple emulsion; a gel; a mousse; an emulsified gel; a dispersion of vesicles; a two-phase lotion; a multiphase lotion; a spray; a free powder; a powder compact; a cast powder; or an anhydrous paste.

20. The composition according to claim 19, wherein the dispersion of vesicles is a dispersion of lipid vesicles.

21. The composition according to claim 1, wherein the composition is in the form of a product to be applied to at least one of mucous membranes, semi-mucous membranes, and keratin tissues.

22. The composition according to claim 21, wherein the composition is in the form of a product to be applied to skin and superficial body growths.

23. The composition according to claim 22, wherein the superficial body growths are chosen from nails, eyelashes, eyebrows, body hair, and head hair.

24. The composition according to claim 1, wherein the at least one substituent is chosen from halogens, hydroxyl radicals, amino radicals, nitrile radicals, dialkylsiloxane radicals, trialkylsilane radicals, and units chosen from linear, branched and cyclic, saturated and unsaturated hydrocarbon-based radicals comprising from 1 to 36 carbon atoms, wherein the units may optionally comprise from 1 to 12 hetero atoms chosen from O, N, S and Si and wherein the units may optionally be substituted by at least one substituent.

25. A make-up product for body skin lips or for keratin fibres, a care product for facial or body skin, an antisun composition, an artificial tanning composition, or a hair composition comprising at least one ingredient chosen from compounds of formula (I) and salts thereof: 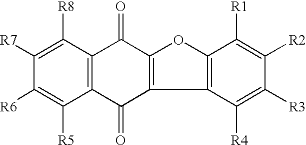
in which the radicals R1 to R8, which may be identical or different, are chosen from:
a hydrogen atom;
a halogen atom;
a hydroxyl radical;
an amino radical —NRR′, wherein R and R′, which may be identical or different, are each a unit chosen from a hydrogen atom and linear, branched and cyclic, saturated and unsaturated hydrocarbon-based radicals comprising from 1 to 36 carbon atoms;
a nitro radical;
an alkylamido radical —NH—CO—R″ wherein R″ is a unit chosen from linear, branched and cyclic, saturated and unsaturated hydrocarbon-based radicals comprising from 1 to 36 carbon atoms;
a ureido radical —NH—CO—NH—R′″ wherein R′″ is a unit chosen from a hydrogen atom and linear, branched and cyclic, saturated and unsaturated hydrocarbon-based radicals comprising from 1 to 36 carbon atoms;
an alkylurethane radical of formula —O—CO—NHR″″ wherein R″″ is a unit chosen from linear, branched and cyclic, saturated and unsaturated hydrocarbon-based radicals comprising from 1 to 36 carbon atoms;
a dialkylsiloxane radical of formula: 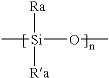
in which
n is an integer ranging from 1 to 12;
Ra and R′a, which may be identical or different, are each a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, wherein the unit may be optionally substituted with at least one substituent;
a trialkylsilane radical of formula: 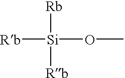
in which Rb, R′b and R″b, which may be identical or different, are each a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, wherein the unit may be optionally substituted with at least one substituent; and
a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, wherein the unit may be optionally substituted with at least one substituent;
wherein at least one of the pair of radicals R1 with R2 and R3 with R4, together with the carbon atoms to which they are attached, may optionally form a ring chosen from a saturated ring and an unsaturated ring, the ring comprising from 5 to 6 carbon atoms in total, wherein the ring may optionally comprise at least one hetero atom chosen from O, N and S, and, wherein the ring may be optionally substituted with at least one substituent;
wherein the radicals R2 and R3, together with the carbon atoms to which they are attached, may optionally form a ring chosen from a saturated ring and an unsaturated ring, the ring comprising from 5 to 6 carbon atoms in total, wherein the ring may optionally comprise at least one hetero atom chosen from O, N and S, and, wherein the ring may be optionally substituted with at least one substituent.

26. The product according to claim 25, wherein the keratin fibres are chosen from nails, eyelashes, eyebrows, and hair.

27. The product according to claim 25, wherein the make-up product is chosen from a foundation, a tinted cream, a face power, an eyeshadow, a free powder, a compact power, a concealer stick, a coverstick, an eyeliner, a mascara, a lipstick, a nail varnish, and a make-up composition for hair.

28. The product according to claim 25, wherein the composition is in the form of a care product for the scalp or a make-up product for the skin of the face.

29. The product according to claim 28, wherein the care product is a care composition for the face chosen from a day, night, anti-wrinkle, and moisturising composition.

30. The product according to claim 29, wherein the care product is a matte-effect composition for the face.

31. The product according to claim 25, wherein the hair composition is chosen from a styling cream, a styling gel, an oxidation dye composition, a direct dye composition, and a colouring shampoo.

32. The product according to claim 25, wherein the at least one substituent is chosen from halogens, hydroxyl radicals, amino radicals, nitrile radicals, dialkylsiloxane radicals, trialkylsilane radicals, and units chosen from linear, branched and cyclic, saturated and unsaturated hydrocarbon-based radicals comprising from 1 to 36 carbon atoms, wherein the units may optionally comprise from 1 to 12 hetero atoms chosen from O, N, S and Si and wherein the units may optionally be substituted by at least one substituent.

33. A method for making a coloured cosmetic composition, comprising including in a cosmetic composition at least one colouring agent chosen from compounds of formula (I) and salts thereof: 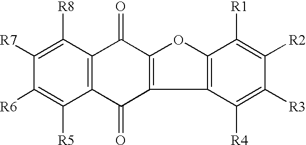
in which the radicals R1 to R8, which may be identical or different, are chosen from:
a hydrogen atom;
a halogen atom;
a hydroxyl radical;
an amino radical —NRR′, wherein R and R′, which may be identical or different, are each a unit chosen from a hydrogen atom and linear, branched and cyclic, saturated and unsaturated hydrocarbon-based radicals comprising from 1 to 36 carbon atoms;
a nitro radical;
an alkylamido radical —NH—CO—R″ wherein R″ is unit chosen from linear, branched and cyclic, saturated and unsaturated hydrocarbon-based radicals comprising from 1 to 36 carbon atoms;
a ureido radical —NH—CO—NH—R′″ wherein R′″ is a unit chosen from a hydrogen atom and linear, branched and cyclic, saturated and unsaturated hydrocarbon-based radicals comprising from 1 to 36 carbon atoms;
an alkylurethane radical of formula —O—CO—NHR″″ wherein R″″ is a unit chosen from linear, branched and cyclic, saturated and unsaturated hydrocarbon-based radicals comprising from 1 to 36 carbon atoms;
a dialkylsiloxane radical of formula: 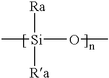
in which
n is an integer ranging from 1 to 12;
Ra and R′a, which may be identical or different, are each a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, wherein the unit may be optionally substituted with at least one substituent;
a trialkylsilane radical of formula: 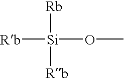
in which Rb, R′b and R″b, which may be identical or different, are each a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, wherein the unit may be optionally substituted with at least one substituent; and
a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, wherein the unit may be optionally substituted with at least one substituent;
wherein at least one of the pair of radicals R1 with R2 and R3 with R4, together with the carbon atoms to which they are attached, may optionally form a ring chosen from a saturated ring and an unsaturated ring, the ring comprising from 5 to 6 carbon atoms in total, wherein the ring may optionally comprise at least one hetero atom chosen from O, N and S, and, wherein the ring may be optionally substituted with at least one substituent;
wherein the radicals R2 and R3, together with the carbon atoms to which they are attached, may optionally form a ring chosen from a saturated ring and an unsaturated ring, ring comprising from 5 to 6 carbon atoms in total, wherein the ring may optionally comprise at least one hetero atom chosen from O, N and S, and, wherein the ring may be optionally substituted with at least one substituent.

34. The method according to claim 33, wherein the composition further comprises a cosmetically acceptable medium.

35. The method according to claim 33, wherein the halogen atom is chosen from chlorine, bromine, iodine, and fluorine.

36. The method according to claim 33, wherein the cosmetic composition is in the form of a product to be applied to at least one of mucous membranes, semi-mucous membranes, and keratin tissues.

37. The method according to claim 36, wherein the cosmetic composition is in the form of:
a make-up product for skin of a face, a body, lips or for keratin fibres;
a care product for facial or body skin;
an antisun composition or artificial tanning composition; or
a hair composition.

38. The method according to claim 37, wherein the keratin fibres are chosen from nails, eyelashes, eyebrows, and hair.

39. The method according to claim 37, wherein the make-up product is chosen from a foundation, a tinted cream, a face powder, an eyeshadow, a free powder, a compact powder, a concealer stick, a coverstick, an eyeliner, a mascara, a lipstick, a nail varnish, and a make-up composition for hair.

40. The method according to claim 37, wherein the composition is in the form of a care product for the scalp.

41. The method according to claim 40, wherein the care product is a care composition for the face chosen from a day, night, anti-wrinkle, and moisturising composition.

42. The method according to claim 41, wherein the care product is a matte-effect composition for the face.

43. The method according to claim 37, wherein the hair composition is chosen from a styling cream, a styling gel, an oxidation dye composition, a direct dye composition, and a colouring shampoo.

44. The method according to claim 33, wherein the at least one substituent is chosen from halogens, hydroxyl radicals, amino radicals, nitrile radicals, dialkylsiloxane radicals, trialkylsilane radicals, and units chosen from linear, branched and cyclic, saturated and unsaturated hydrocarbon-based radicals comprising from 1 to 36 carbon atoms, wherein the units may optionally comprise from 1 to 12 hetero atoms chosen from O, N, S and Si and wherein the units may optionally be substituted by at least one substituent.

45. A compound corresponding to one of the formulae (IIa) to (VIa) below or a salt thereof: 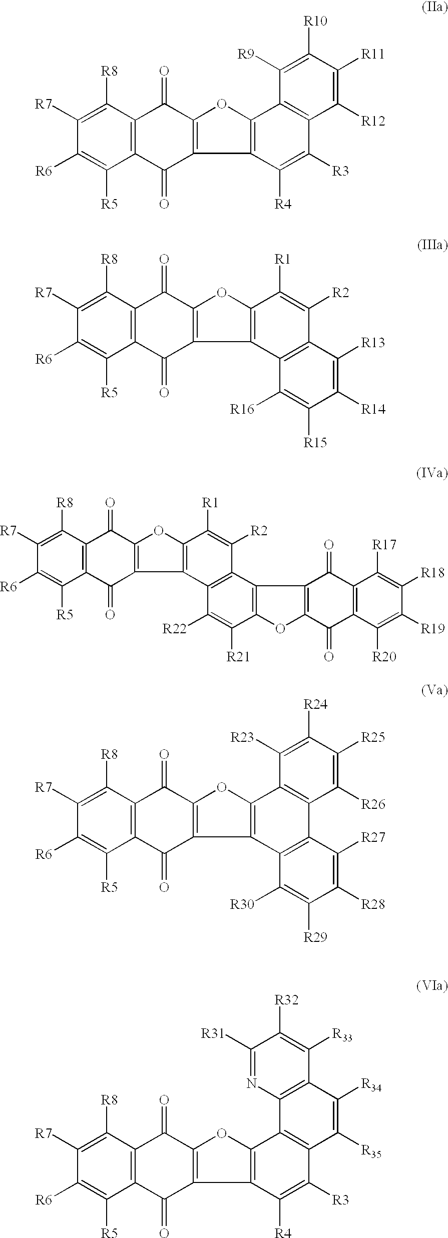
in which: the radicals R1 to R35, which may be identical or different, are chosen from:
a hydrogen atom;
a halogen atom;
a hydroxyl radical;
an amino radical —NRR′ wherein R and R′, which may be identical or different, are each a unit chosen from a hydrogen atom and linear, branched and cyclic, saturated and unsaturated hydrocarbon-based radicals comprising from 1 to 36 carbon atoms;
a nitro radical;
an alkylamido radical —NH—CO—R″ wherein R″ is a unit chosen from linear, branched and cyclic, saturated and unsaturated hydrocarbon-based radicals comprising from 1 to 36 carbon atoms;
a ureido radical —NH—CO—NH—R′″ wherein R′″ is a unit chosen from a hydrogen atom and linear, branched and cyclic, saturated and unsaturated hydrocarbon-based radicals comprising from 1 to 36 carbon atoms;
an alkylurethane radical of formula —O—CO—NHR″″ wherein R″″ is a unit chosen from linear, branched and cyclic, saturated and unsaturated hydrocarbon-based radicals comprising from 1 to 36 carbon atoms;
a dialkylsiloxane radical of formula: 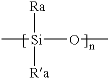
in which
n is an integer ranging from 1 to 12;
Ra and R′a, which may be identical or different, are each a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, wherein the unit may optionally be substituted with at least one substituent;
a trialkylsilane radical of formula: 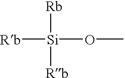
in which Rb, R′b and R″b, which may be identical or different, are each a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, and wherein the unit may optionally be substituted by at least one substituent; and
a unit chosen from linear, branched and cyclic, saturated and unsaturated hydrocarbon-based radicals comprising from 1 to 36 carbon atoms, wherein the unit may optionally comprise from 1 to 12 hetero atoms chosen from O, N, S and Si and, and wherein the unit may optionally be substituted by at least one substituent; wherein at least one of the pair of radicals R1 with R2 and R3 with R4, together with the carbon atoms to which they are attached, may optionally form a ring chosen from a saturated ring and an unsaturated ring, the ring comprising from 5 to 6 carbon atoms in total, wherein the ring may optionally comprise at least one hetero atom chosen from O, N and S, and wherein the ring may optionally be substituted with at least one substituent;
wherein the radicals R2 and R3, together with the carbon atoms to which they are attached, may optionally form a ring chosen from a saturated ring and an unsaturated ring, the ring comprising from 5 to 6 carbon atoms in total, wherein the ring may optionally comprise at least one hetero atom chosen from O, N and S, and wherein the ring may optionally be substituted with at least one substituent, with the exception of the following compounds and salts:
of formula (IIa) or (IIIa) or (Va) in which all the radicals are H;
of formula (IIa) in which R3=OH and all the other radicals are H;
of formula (IIa) in which R3=OCH₃ and all the other radicals are H;
of formula (IIa) in which R10=OH and all the other radicals are H;
of formula (IIa) in which R11=OH and all the other radicals are H;
of formula (IIIa) in which all the radicals are H;
of formula (IIIa) in which R13=OCH₃ and all the other radicals are H;
of formula (IIIa) in which R13=OH and all the other radicals are H;
of formula (IIIa) in which R₁=OH and all the other radicals are H;
of formula (IIIa) in which R2=OH and all the other radicals are H;
of formula (IIIa) in which R1=OCH₃ and all the other radicals are H;
of formula (IIa) or (IIIa) in which R₅=NO₂ and all the other radicals are H;
of formula (IIa) or (IIIa) in which R₈=NO₂ and all the other radicals are H;
of formula (IIa) or (IIIa) in which R₅=NH₂ and all the other radicals are H;
of formula (IIa) or (IIIa) in which R₈=NH₂ and all the other radicals are H;
of formula (IIa) or (IIIa) in which R₅ is benzamido and all the other radicals are H;
of formula (IIa) or (IIIa) in which R₈ is benzamido and all the other radicals are H;
of formula (IIIa) in which R₈=Br and all the other radicals are H;
of formula (IIIa) in which R₈=p-tolylsulphon-amido and all the other radicals are H;
of formula (IIIa) in which R₁=OCH₃ and all the other radicals are H;
of formula (IIIa) in which R₂=O(CH₂)₂—N(CH₃)₂H.Cl and all the other radicals are H;
of formula (IIIa) in which R₂=O(CH₂)₃—N(CH₃)₂H.Cl and all the other radicals are H;
of formula (IIIa) in which R₁ is chosen from —CONH-(2′-pyridyl), —CONH-(2′-pyrimidinyl), —CONH-(2′-thiazolyl), —CONH-(3′(H-1,2,4-triazolyl)) and —CONH-phenyl and all the other radicals are H;
of formula (IIIa) in which R₁ is chosen from —CONH-(2′-methylphenyl), —CONH-(4′-cyanophenyl), —CONH-(2′(3′-methoxypyridyl)), and —CONH-(4′-methoxyphenyl), R14 and R15 together are —CH═CH—CH═CH— and all the other radicals are H; and
of formula (IIIa) in which R₁ has the following formula: 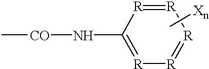
in which: R is chosen from N and CH, wherein from 1 to 3 radicals R=N, X is chosen from H, CH₃, C₂H₅, NO₂, OCH₃, CN, SO₂NH₂, CO₂CH₃, CO₂C₂H₅, SO₂NHC₆H₅, Cl, F, Br and I, and n is a positive integer ranging from 1 to 4.

46. A compound according to claim 45, wherein the compound is chosen from:
5-chloro-dinaphto(1,2-b:2′,3′-d)furan-7,12-dione,
5-isopropyloxy-dinaphto(1,2-b:2′,3′-d)furan-7,12-dione,
2-méthoxy-dinaphto (2,1-b:2′,3′-d)furan-8,13-dione,
3-bromo-dinaphto(2,1-b:2′,3′-d)furan-8,13-dione,
5-hexyloxy-dinaphto(1,2-b:2′,3′-d)furan-7,12-dione,
5-(cholest-5-en-3β-ol)-dinaphto(1,2-b:2′,3′-d)furan-7,12-dione,
(dinaphto(2,1-b:2′,3′-d)furan-8,13-dione)(3,4-b)naphtho(2′,3′-d)furan-5,14-dione,
3-méthoxy-dinaphtho(2,1-b:2′,3′-d)furan-8,13-dione, and
naphtho(2,3-b)-5-azo-phenanthro(3′,4′-d)furan-10,15-dione.

47. The compound according to claim 45, wherein the at least one substituent is chosen from halogens, hydroxyl radicals, amino radicals, nitrile radicals, dialkylsiloxane radicals, trialkylsilane radicals, and units chosen from linear, branched and cyclic, saturated and unsaturated hydrocarbon-based radicals comprising from 1 to 36 carbon atoms, wherein the units may optionally comprise from 1 to 12 hetero atoms chosen from O, N, S and Si and wherein the units may optionally be substituted by at least one substituent.

48. A compound having the following formula: 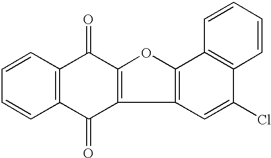
and having the following X-ray diffraction spectrum:

| | | Intensity | |
|---|---|---|---|
| ANGLE 2θ (°) | d (Angstrom) | (count) | Intensity (%) |
| 8.858 | 9.97422 | 1212 | 91.4 |
| 10.545 | 8.38269 | 974 | 73.5 |
| 12.759 | 6.93251 | 1326 | 100 |
| 14.369 | 6.15885 | 366 | 27.6 |
| 15.21 | 5.8203 | 194 | 14.6 |
| 17.439 | 5.08119 | 159 | 12 |
| 17.822 | 4.97271 | 143 | 10.8 |
| 19.769 | 4.48716 | 252 | 19 |
| 21.228 | 4.18188 | 173 | 13 |
| 21.813 | 4.07116 | 126 | 9.5 |
| 22.346 | 3.97513 | 295 | 22.2 |
| 24.67 | 3.60578 | 247 | 18.6 |
| 24.927 | 3.56909 | 209 | 15.8 |
| 25.992 | 3.42522 | 858 | 64.7 |
| 26.525 | 3.3576 | 393 | 29.6 |
| 27.151 | 3.28166 | 692 | 52.2 |
| 28.096 | 3.17331 | 111 | 8.4 |
| 29.412 | 3.03425 | 100 | 7.5 |

49. A compound having the following formula: 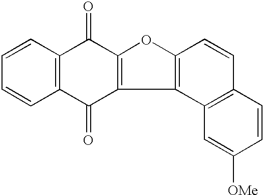
and having the following X-ray diffraction spectrum:

| | | Intensity | |
|---|---|---|---|
| ANGLE 2θ (°) | d (Angstrom) | (count) | Intensity (%) |
| 7.124 | 12.39813 | 1065 | 69.8 |
| 10.087 | 8.76195 | 1055 | 69.1 |
| 10.694 | 8.26606 | 1526 | 100 |
| 12.37 | 7.14936 | 890 | 58.3 |
| 13.022 | 6.79302 | 273 | 17.9 |
| 14.341 | 6.17099 | 453 | 29.7 |
| 14.784 | 5.98695 | 572 | 37.5 |
| 16.572 | 5.34479 | 214 | 14 |
| 16.696 | 5.30553 | 221 | 14.5 |
| 17.8 | 4.97885 | 198 | 13 |
| 18.612 | 4.76353 | 166 | 10.9 |
| 19.125 | 4.63688 | 304 | 19.9 |
| 19.56 | 4.53465 | 251 | 16.4 |
| 20.39 | 4.35189 | 223 | 14.6 |
| 20.653 | 4.2971 | 309 | 20.2 |
| 22.245 | 3.99296 | 337 | 22.1 |
| 23.524 | 3.77866 | 132 | 8.7 |
| 24.553 | 3.62271 | 168 | 11 |
| 25.317 | 3.51502 | 960 | 56.4 |
| 25.901 | 3.4371 | 1255 | 82.2 |
| 26.2 | 3.39852 | 819 | 53.7 |
| 27.568 | 3.23294 | 393 | 25.8 |
| 27.678 | 3.22028 | 420 | 27.5 |
| 29.622 | 3.01328 | 178 | 11.7 |

50. A compound having the following formula: 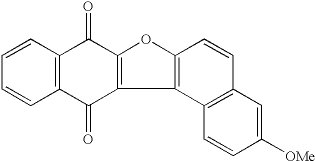
and having the following X-ray diffraction spectrum:

| | | Intensity | |
|---|---|---|---|
| ANGLE 2θ (°) | d (Angstrom) | (count) | Intensity (%) |
| 7.02 | 12.5816 | 1800 | 73.2 |
| 10.555 | 8.3741 | 621 | 25.2 |
| 11.834 | 7.47227 | 2460 | 100 |
| 12.213 | 7.24081 | 976 | 39.7 |
| 12.926 | 6.89616 | 1812 | 73.7 |
| 14.147 | 6.2554 | 267 | 10.9 |
| 14.72 | 6.01306 | 201 | 8.2 |
| 15.813 | 5.59984 | 1474 | 59.9 |
| 17.684 | 5.01117 | 223 | 9.1 |
| 18.403 | 4.81697 | 234 | 9.5 |
| 19.778 | 4.4851 | 118 | 4.8 |
| 21.31 | 4.16604 | 145 | 5.9 |
| 23.051 | 3.8552 | 148 | 6 |
| 23.874 | 3.72413 | 840 | 34.1 |
| 24.184 | 3.67713 | 702 | 28.5 |
| 24.963 | 3.56407 | 360 | 14.6 |
| 25.179 | 3.53403 | 395 | 16.1 |
| 25.731 | 3.4594 | 248 | 10.1 |
| 26.28 | 3.38832 | 589 | 23.9 |
| 26.693 | 3.33691 | 731 | 29.7 |
| 27.218 | 3.27369 | 431 | 17.5 |
| 27.872 | 3.19828 | 468 | 19 |
| 28.62 | 3.1164 | 251 | 10.2 |
| 29.469 | 3.02855 | 129 | 5.2 |
